(19) United States
(12) Patent Application Publication
(10) Pub. No.: US 2026/0244122 A1
(43) Pub. Date: Aug. 20, 2026

(54) ELECTROPHOTOGRAPHIC PHOTOSENSITIVE MEMBER, PROCESS CARTRIDGE, AND ELECTROPHOTOGRAPHIC APPARATUS

(71) Applicant: CANON KABUSHIKI KAISHA, Tokyo (JP)

(72) Inventors: ERI FUJISAWA, Chiba (JP); KOICHI NAKATA, Tokyo (JP); IKUYO KUROIWA, Tokyo (JP); NAOHIRO TAKAHASHI, Chiba (JP)

(21) Appl. No.: 19/535,142

(22) Filed: Feb. 10, 2026

(30) Foreign Application Priority Data
Feb. 17, 2025 (JP) 2025-023469

(51) Int. Cl. G03G 5/06; C07C 219/32; G03G 21/18

(52) U.S. Cl. CPC G03G 5/0618; C07C 219/32; G03G 21/1814; G03G 2215/00957; G03G 2215/00962

(57) ABSTRACT

Provided is an electrophotographic photosensitive member including a surface layer having excellent abrasion resistance and suppressing image smearing. The electrophotographic photosensitive member includes a surface layer containing a polymerized product of a polymerizable charge-transporting compound. In dynamic viscoelasticity measurement, the surface layer has a storage elastic modulus G′ and a loss elastic modulus G″ satisfying a relationship of G′>G″ at normal temperature, and has a peak top of Tan δ in a temperature range of 85° C. or more and 100° C. or less.

### BACKGROUND

### Field of the Technology

The present disclosure relates to an electrophotographic photosensitive member, a process cartridge, and an electrophotographic apparatus.

### Description of the Related Art

An organic electrophotographic photosensitive member (hereinafter referred to as “electrophotographic photosensitive member”) to be mounted onto an electrophotographic apparatus has been required to achieve both of an improvement in mechanical durability (abrasion resistance) and suppression of image smearing, which is an image defect, for the purposes of lengthening the lifetime of the electrophotographic photosensitive member and improving image quality at the time of repeated use thereof.

An example of a technology of improving the abrasion resistance of the electrophotographic photosensitive member is a method including incorporating a thiol compound into a surface layer of the electrophotographic photosensitive member to improve the mechanical strength of the surface layer and impart flexibility thereto. In Japanese Patent Laid-Open No. 2012-159521, there is a disclosure of a technology of improving the mechanical strength of a surface layer and imparting flexibility and toughness thereto by incorporating a compound having 4 or more primary thiol groups and a compound having 2 or more secondary thiol groups into the surface layer.

In addition, an example of a technology of suppressing image smearing is a method including adopting a methacryloyl group as a chain polymerizable functional group of a resin to be used in a surface layer of an electrophotographic photosensitive member. In Japanese Patent Laid-Open No. 2013-137490, there is a disclosure of a technology of achieving excellent potential stability at the time of repeated use of a photosensitive member and suppressing image smearing by forming a surface layer including a compound having a methacryloyl group as a chain polymerizable functional group and having 2 functional groups.

However, in the technology disclosed in Japanese Patent Laid-Open No. 2012-159521, a further improvement in abrasion resistance has been required to meet the demand for higher durability. In addition, in the technology disclosed in Japanese Patent Laid-Open No. 2013-137490, while a surface layer capable of suppressing image smearing is obtained, abrasion resistance is degraded in some cases. Accordingly, there has been room for improvement in achieving both the abrasion resistance and suppression of image smearing of the electrophotographic photosensitive member.

### SUMMARY

One aspect of the present disclosure is directed to providing an electrophotographic photosensitive member having high abrasion resistance and suppressing image smearing. Another aspect of the present disclosure is directed to providing a process cartridge including the electrophotographic photosensitive member, and an electrophotographic apparatus including the process cartridge.

According to one aspect of the present disclosure, there is provided an electrophotographic photosensitive member including a surface layer containing a polymerized product of at least one kind of polymerizable charge-transporting compound selected from the group consisting of: a compound represented by the formula (CT-1); and a compound represented by the formula (CT-2), wherein, in dynamic viscoelasticity measurement, the surface layer has a storage elastic modulus G′ and a loss elastic modulus G″ satisfying a relationship of G′>G″ at normal temperature, and has a peak top of Tan δ in a temperature range of 85° C. or more and 100° C. or less:

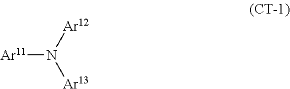

in the formula (CT-1), Ar¹¹ to Ar¹³ each independently represent a substituted aryl group or an unsubstituted aryl group, and a substituent that the substituted aryl group may have is an alkyl group having 1 or more and 4 or less carbon atoms, or a monovalent functional group represented by the formula (P-1), provided that the compound represented by the formula (CT-1) has at least one monovalent functional group represented by the formula (P-1):

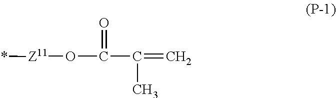

in the formula (P-1), Z¹¹ represents an alkylene group having 1 or more and 6 or less carbon atoms, and * represents a bonding site with a structure represented by the formula (CT-1) or (CT-2):

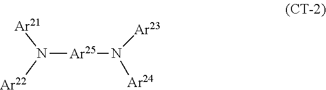

in the formula (CT-2), Ar²¹ to Ar²⁴ each independently represent a substituted aryl group or an unsubstituted aryl group, Ar²⁵ represents a substituted arylene group or an unsubstituted arylene group, a substituent that the substituted aryl group may have is an alkyl group having 1 or more and 4 or less carbon atoms, or a monovalent functional group represented by the formula (P-1), and a substituent that the substituted arylene group may have is an alkyl group having 1 or more and 4 or less carbon atoms, or a monovalent functional group represented by the formula (P-1), provided that the compound represented by the formula (CT-2) has at least one monovalent functional group represented by the formula (P-1).

In addition, in the electrophotographic photosensitive member, the storage elastic modulus G′ is 1,000 Pa or more, and the loss elastic modulus G″ is 1,000 Pa or more.

In addition, in the electrophotographic photosensitive member, the surface layer contains a polymerized product of a composition containing the polymerizable charge-transporting compound and a thiol compound, wherein, when a content of the polymerizable charge-transporting compound represented by each of the formulae (CT-1) and (CT-2) in the composition is represented by WCT, and a content of the thiol compound therein is represented by WT, the WCT and the WT satisfy the formula (1).

$$0.01 \leq WT / WCT \leq 0.07 \qquad ( 1 )$$

In addition, in the electrophotographic photosensitive member, the thiol compound is a compound represented by the formula (T-1):

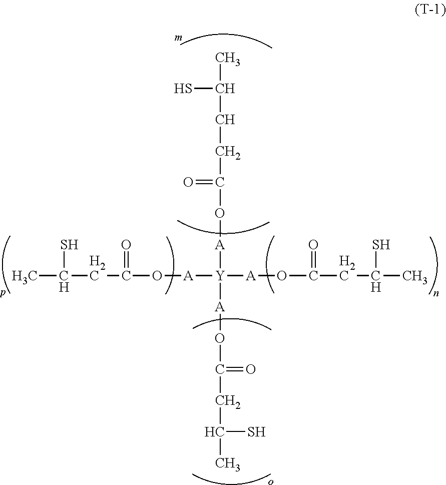

in the formula (T-1), Y represents a carbon atom, or a heterocycle having 2 or more nitrogen atoms, and A represents a single bond, a hydrogen atom, or a linear alkylene group having 1 or more and 3 or less carbon atoms, “m”, “n”, “o”, and “p” each represent an integer of 0 or 1, and m+n+o+p is 2 or more.

Features of the present disclosure will become apparent from the following description of embodiments with reference to the attached drawings. The following description of embodiments is described by way of example.

### BRIEF DESCRIPTION OF THE DRAWINGS

FIG. 1 is a schematic view for illustrating an example of a configuration of an electrophotographic photosensitive member of the present disclosure.

FIG. 2 is a view for illustrating an example of a polishing machine using a polishing sheet.

FIG. 3 is a view for illustrating an example of a schematic configuration of a process cartridge including the electrophotographic photosensitive member of the present disclosure, and an electrophotographic apparatus including the process cartridge.

FIG. 4 is a schematic view for illustrating an example of a process cartridge including the electrophotographic photosensitive member of the present disclosure.

FIG. 5 is a schematic view for illustrating an example of an electrophotographic apparatus including the electrophotographic photosensitive member of the present disclosure.

### DESCRIPTION OF THE EMBODIMENTS

In the present disclosure, the description “XX or more and YY or less” or “from XX to YY” representing a numerical range means a numerical range including a lower limit and an upper limit that are end points unless otherwise stated. In addition, when the numerical ranges are described in stages, the upper and lower limits of each numerical range may be freely combined. The present disclosure is described in detail below by way of exemplary embodiments.

The inventors have made investigations, and as a result, have found that an electrophotographic photosensitive member including a surface layer containing a polymerized product of at least one kind of polymerizable charge-transporting compound selected from the group consisting of: a compound represented by the formula (CT-1); and a compound represented by the formula (CT-2), wherein, in dynamic viscoelasticity measurement, the surface layer has a storage elastic modulus G′ and a loss elastic modulus G″ satisfying a relationship of G′>G″ at normal temperature, and has a peak top of Tan δ in a temperature range of 85° C. or more and 100° C. or less, has abrasion resistance and suppresses image smearing:

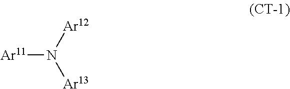

in the formula (CT-1), Ar¹¹ to Ar¹³ each independently represent a substituted aryl group or an unsubstituted aryl group, and a substituent that the substituted aryl group may have is an alkyl group having 1 or more and 4 or less carbon atoms, or a monovalent functional group represented by the formula (P-1), provided that the compound represented by the formula (CT-1) has at least one monovalent functional group represented by the formula (P-1):

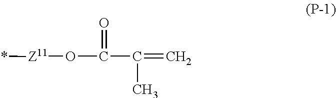

in the formula (P-1), Z¹¹ represents an alkylene group having 1 or more and 6 or less carbon atoms, and * represents a bonding site with the structure represented by the formula (CT-1) or (CT-2):

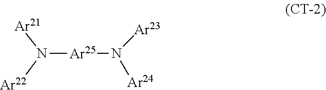

in the formula (CT-2), Ar²¹ to Ar²⁴ each independently represent a substituted aryl group or an unsubstituted aryl group, Ar²⁵ represents a substituted arylene group or an unsubstituted arylene group, a substituent that the substituted aryl group may have is an alkyl group having 1 or more and 4 or less carbon atoms, or a monovalent functional group represented by the formula (P-1), and a substituent that the substituted arylene group may have is an alkyl group having 1 or more and 4 or less carbon atoms, or a monovalent functional group represented by the formula (P-1), provided that the compound represented by the formula (CT-2) has at least one monovalent functional group represented by the formula (P-1).

The inventors have assumed the reasons why the electrophotographic photosensitive member of the present disclosure is excellent in abrasion resistance and excellent in suppression of image smearing to be as described below.

Image smearing is liable to occur with an electrophotographic photosensitive member including a surface layer containing a charge-transporting compound in which the polymerizable functional group is an acrylic group. This is conceived to be because when an acrylic group is subjected to discharge, a discharge product derived from oxalic acid formed through its interaction with oxygen or water in the atmosphere accumulates on the surface of a drum to reduce the surface resistance of the drum, and hence image smearing is liable to occur.

The inventors have made investigations, and as a result, have found that image smearing can be suppressed by incorporating, into the surface layer, a polymerized product of a charge-transporting compound represented by each of the formulae (CT-1) and (CT-2), in which the polymerizable functional group is a methacryloyl group. The C═C double bond portion of the methacryloyl group is protected by a methyl group, and hence the interaction of the methacryloyl group with oxygen or water in the atmosphere hardly occurs even when the electrophotographic photosensitive member is subjected to discharge degradation. Thus, it is conceived that the formation of a discharge product derived from oxalic acid is suppressed, and hence image smearing can be suppressed. However, a surface layer containing a polymerized product of a charge-transporting compound in which the polymerizable functional group is a methacryloyl group has abrasion resistance lower than that of a surface layer in which the polymerizable functional group is an acrylic group, and hence there has been room for improvement.

In view of the foregoing, as a result of further investigations by the inventors, it has been found that an electrophotographic photosensitive member excellent in abrasion resistance on its surface can be obtained by adjusting the surface layer to satisfy the relationship of storage elastic modulus (G′)>loss elastic modulus (G″) at normal temperature, and have a peak top of Tan δ in the temperature range of 85° C. or more and 100° C. or less in the dynamic viscoelasticity measurement.

The temperature of the Tan δ peak top, which is a value obtained in dynamic viscoelasticity measurement, is a temperature at which the movement of molecular chains begins to become active, and is a temperature at which molecular expansion/contraction and rotational movement within the molecular chains increase. When molecular expansion/contraction and rotational movement within the molecular chains increase, internal stress can be dispersed. Accordingly, it is conceived that when the surface layer satisfies the relationship of storage elastic modulus (G′)>loss elastic modulus (G″) at normal temperature, and the Tan δ peak top temperature is set to a range of temperatures caused by sliding rotation of the electrophotographic photosensitive member and a cleaning blade, that is, a nip portion frictional heat temperature of 85° C. or more and 100° C. or less, the pressure applied from the blade to the electrophotographic photosensitive member can be most dispersed, and the abrasion amount can be suppressed. Examples of a method of adjusting the Tan δ peak top temperature in the dynamic viscoelasticity measurement within the above-mentioned range include a method including adjusting the total number of carbon atoms of the alkyl group and the alkylene group in the substituted aryl group represented by each of Ar¹¹ to Ar¹³ in the formula (CT-1), a method including incorporating a polymerized product of a composition containing a thiol compound represented by the formula (T-1) into the surface layer, and a combination thereof.

For example, in the present disclosure, in the formula (CT-1), Ar¹¹ to Ar¹³ each independently represent a substituted or unsubstituted aryl group, and at least one of Ar¹¹ to Ar¹³ has a monovalent functional group represented by the formula (P-1). The flexibility of an alkyl chain may be adjusted by adjusting the total number of carbon atoms of the alkylene group Z¹¹ in the formula (P-1) and the respective alkyl groups in the aryl groups other than the formula (P-1). In order to adjust the surface layer to satisfy the relationship of storage elastic modulus (G′)>loss elastic modulus (G″) at normal temperature, and have a peak top of Tan δ in the temperature range of 85° C. or more and 100° C. or less in the dynamic viscoelasticity measurement, the total number of carbon atoms is preferably set to 2 or more and 15 or less. When the total number of carbon atoms is set within the above-mentioned range, it is conceived that a pressure applied from a blade to the electrophotographic photosensitive member can be most dispersed by the flexibility of the alkyl chain, the abrasion amount is suppressed, and an electrophotographic photosensitive member having high abrasion resistance and suppressing image smearing can be obtained.

Further, in the present disclosure, it is preferred to incorporate a polymerized product of a composition containing the thiol compound represented by the formula (T-1) into the surface layer. When the polymerized product of the composition containing the thiol compound represented by the formula (T-1) is incorporated into the surface layer, the polymerizable functional group of the charge-transporting compound and the thiol compound undergo a thiol-ene reaction to form a C—S bond in a cured film serving as the surface layer. The C—S bond has bond energy lower than that of a C—C bond, and hence stability within the molecule is lowered, and the Tan δ peak top can be adjusted. Specifically, when the content of the polymerizable charge-transporting compound represented by each of the formulae (CT-1) and (CT-2) in the composition of the surface layer is represented by WCT, and the content of the thiol compound therein is represented by WT, the incorporation of the polymerized product into the surface layer so that WCT and WT satisfy the formula (1):

$$0.01 \leq WT / WCT \leq 0.07 \qquad ( 1 )$$

enables the adjustment of the temperature of the Tan δ peak top within the range of 85° C. or more and 100° C. or less. Accordingly, the pressure applied from the blade to the electrophotographic photosensitive member can be most dispersed, and the abrasion amount is suppressed. When WT/WCT is smaller than 0.01, the Tan δ peak top temperature is higher than the nip portion frictional heat temperature, and it is conceived that molecular motion is dull under the nip portion frictional heat temperature. Accordingly, it is conceived that the pressure from the blade to the electrophotographic photosensitive member is not dispersed, the surface layer cracks, and the abrasion amount increases. When WT/WCT is larger than 0.07, the Tan δ peak top temperature is lower than the nip portion frictional heat temperature, and molecular motion becomes more active under the nip portion frictional heat temperature. The molecular motion is not limited to molecular expansion/contraction and rotational movement within the molecule, but the entire molecular chain begins to move and have fluidity. It is conceived that when fluidity increases, the amount of deformation of the surface layer increases, and the abrasion amount increases. Accordingly, when the thiol compound represented by the formula (T-1) is incorporated so that the formula (1) is satisfied, an electrophotographic photosensitive member having high abrasion resistance and suppressing image smearing is obtained:

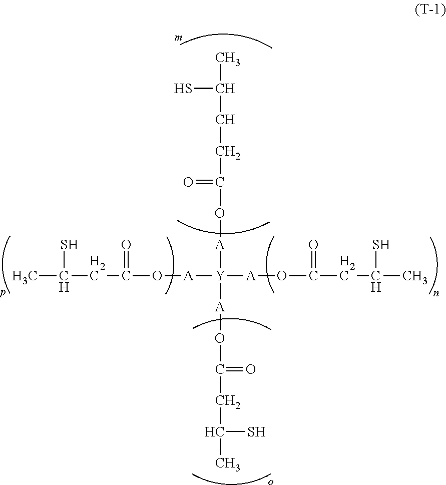

in the formula (T-1), Y represents a carbon atom, or a heterocycle having 2 or more nitrogen atoms, A represents a single bond, a hydrogen atom, or a linear alkylene group having 1 or more and 3 or less carbon atoms, “m”, “n”, “o”, and “p” each represent an integer of 0 or 1, and m+n+0+p is 2 or more.

The relationship between the storage elastic modulus G′ and the loss elastic modulus G″ of the surface layer is G′>G″ at normal temperature. Further, it is preferred that the storage elastic modulus G′ be 1,000 Pa or more, and the loss elastic modulus G″ be 1,000 Pa or more. In addition, it is more preferred that both the storage elastic modulus G′ and the loss elastic modulus G″ be 10,000 Pa or more.

### <Charge-Transporting Compound Represented by Formula (CT-1)>

In the surface layer of the electrophotographic photosensitive member of the present disclosure, when the surface layer of the electrophotographic photosensitive member is formed, the compounds each represented by the formula (CT-1) may be used alone or in combination thereof. Specific examples of the compound represented by the formula (CT-1) of the present disclosure are given below, but the present disclosure is not limited thereto.

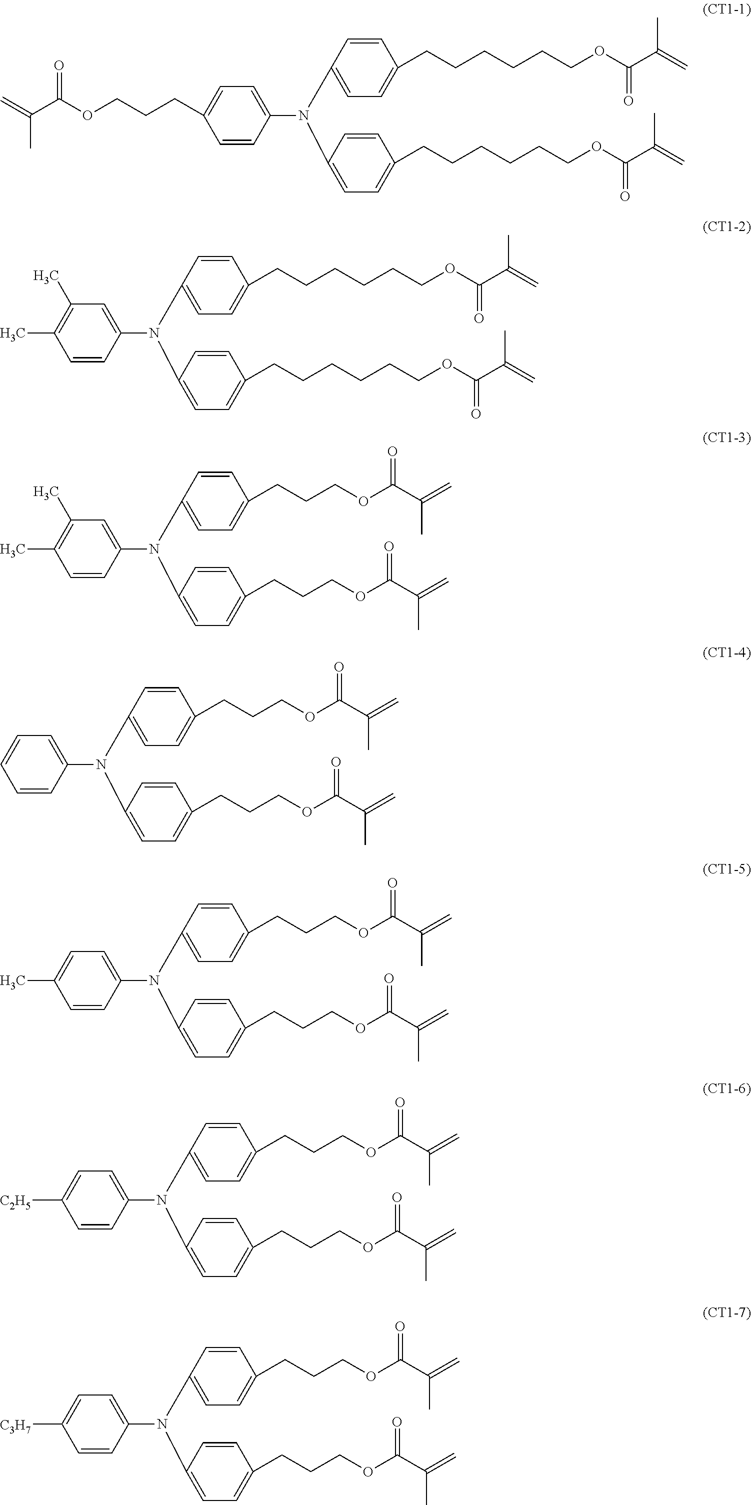

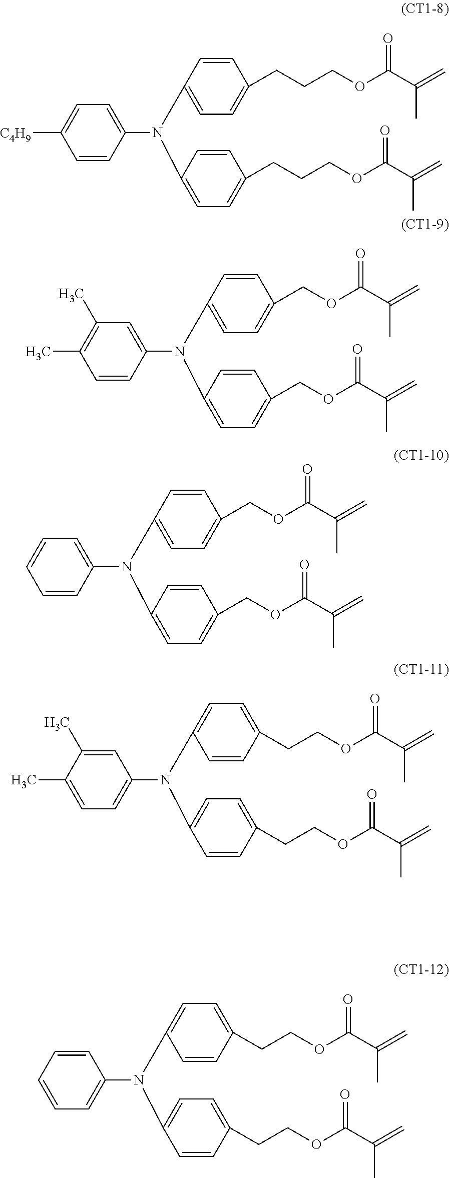

### <Charge-Transporting Compound Represented by Formula (CT-2)>

In the surface layer of the electrophotographic photosensitive member of the present disclosure, when the surface layer of the electrophotographic photosensitive member is formed, the compounds each represented by the formula (CT-2) may be used alone or in combination thereof. Specific examples of the compound represented by the formula (CT-2) of the present disclosure are given below, but the present disclosure is not limited thereto.

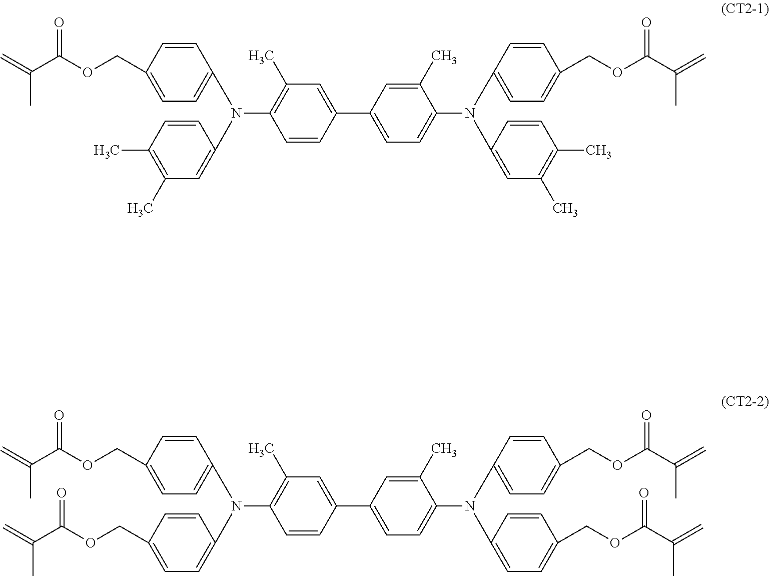

### <Measurement of Tan δ Peak Top Temperature in Dynamic Viscoelasticity Measurement>

In Examples of the present disclosure, a rotational flat plate-type rheometer “ARES” (manufactured by TA Instruments) was used in the measurement of the storage elastic modulus and Tan δ of the surface layer of the electrophotographic photosensitive member. The measurement was performed by using a sample of the surface layer alone obtained by scraping off the surface layer of the electrophotographic photosensitive member with a razor and rubbing the back surface of the scraped film with a cotton swab soaked in xylene. The measurement is performed under the following conditions.
(1) A parallel plate having a diameter of 7.9 mm is used.
(2) The frequency (Frequency) is set to 6.28 rad/see (1.0 Hz).
(3) The initial value of applied strain (Strain) is set to 0.1%.
(4) Measurement is performed between 30° C. and 200° C. at a temperature increase rate (Ramp Rate) of 2.0° C./min. The measurement is performed under the following setting conditions of an automatic adjustment mode. Measurement is performed in an automatic strain adjustment mode (Auto Strain).
(5) The maximum strain (Max Applied Strain) is set to 20.0%.
(6) The maximum torque (Max Allowed Torque) is set to 200.0 g cm, and the minimum torque (Min Allowed Torque) is set to 0.2 g cm.
(7) Strain Adjustment is set to 20.0% of Current Strain. In the measurement, an automatic tension adjustment mode (Auto Tension) is adopted.
(8) Auto Tension Direction is set to Compression.
(9) Initial Static Force is set to 10.0 g, and Auto Tension Sensitivity is set to 40.0 g.
(10) The operating condition of Auto Tension is Sample Modulus of 1.0×10³ Pa or more.

### <Thiol Compound Represented by Formula (T-1)>

In the surface layer of the electrophotographic photosensitive member of the present disclosure, when the surface layer of the electrophotographic photosensitive member is formed, the thiol compounds each represented by the formula (T-1) may be used alone or in combination thereof.

Specifically, KarenzMT (trademark) BD1 (1,4-bis(3-mercaptobutyryloxy) butane, manufactured by Resonac Corporation, a compound having 2 secondary thiol groups), KarenzMT NR1 (1,3,5-tris(3-mercaptobutyryloxyethyl)-1,3,5-triazine-2,4,6(1H,3H,5H)-trione, manufactured by Resonac Corporation, a compound having 3 secondary thiol groups), or KarenzMT PE1 (pentaerythritol tetrakis(3-mercaptobutyrate), manufactured by Resonac Corporation, a compound having 4 secondary thiol groups) is suitably used as the thiol compound.

With regard to the content of the thiol compound represented by the formula (T-1) to be incorporated into the surface layer of the electrophotographic photosensitive member of the present disclosure, from the viewpoint of abrasion resistance, when the content of the polymerizable charge-transporting compound represented by each of the formulae (CT-1) and (CT-2) in the composition is represented by WCT, and the content of the thiol compound therein is represented by WT, WT/WCT is preferably 0.01 or more and 0.03 or less, particularly preferably 0.02.

In addition, the molecular weight of the thiol compound represented by the formula (T-1) is preferably 250 or more and 600 or less, more preferably 260 or more and 570 or less.

### <Measurement of Residual Amount of Thiol Compound>

The residual ratio of the thiol compound to be incorporated into the surface layer of the electrophotographic photosensitive member of the present disclosure was evaluated as described below.

The surface layer at a position of 179 mm from the upper end of the electrophotographic photosensitive member was scraped off with a razor. The scraped piece of the surface layer was subjected to infrared absorption measurement by attenuated total reflectance-Fourier transform infrared spectroscopy, and the IR peak ratio of the residue of the thiol compound (S—H group) that did not contribute to the polymerization reaction was calculated. Spectrum One FT-IR Spectrometer manufactured by PerkinElmer Instruments was used as a Fourier transform infrared spectrometer, and the measurement was performed by using the following prisms: a Ge prism and a KRS-5 prism. When the absorbance of a S—H stretching absorption band derived from the thiol compound and showing an infrared absorption spectrum in the range of from 2,550 cm⁻¹ to 2,600 cm⁻¹ is represented by AT, and the absorbance of a C═O stretching absorption band of an ester group derived from the thiol compound and showing an infrared absorption spectrum in the range of from 1,735 cm-1 to 1,750 cm⁻¹ is represented by AE, AT/AE is preferably 0.001 or less.

For example, when the absorbance of a S—H stretching absorption band of a thiol compound derived from KarenzMT BD1, KarenzMT NR1, or KarenzMT PE1, which is a thiol compound, and showing an infrared absorption spectrum in the range of from 2,550 cm⁻¹ to 2,600 cm⁻¹ is represented by AT, and the absorbance of a C═O stretching absorption band of an ester group of a thiol compound derived from KarenzMT BD1, KarenzMT NR1, or KarenzMT PE1, which is a thiol compound, and showing an infrared absorption spectrum in the range of from 1,735 cm-1 to 1,750 cm⁻¹ is represented by AE, AT/AE is preferably 0.001 or less.

### <Electrophotographic Photosensitive Member>

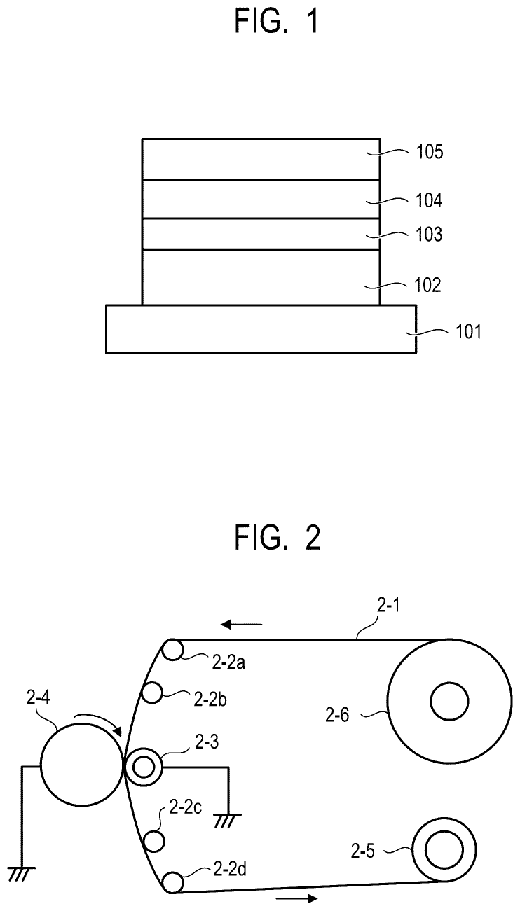

An example of a layer configuration of the electrophotographic photosensitive member of the present disclosure is illustrated in FIG. 1. In FIG. 1, an undercoat layer 102, a charge-generating layer 103, a charge-transporting layer 104, and a protective layer 105 are laminated on a support 101. A photosensitive layer to be described later may include a laminate-type photosensitive layer including the charge-generating layer and the charge-transporting layer, or may include a monolayer-type photosensitive layer containing a charge-generating substance and a charge-transporting substance.

Examples of a method of applying coating liquids of the respective layers include dip coating, spray coating, inkjet coating, roll coating, die coating, blade coating, curtain coating, wire bar coating, and ring coating. Of those, dip coating is preferred from the viewpoints of efficiency and productivity.

The configuration of the electrophotographic photosensitive member of the present disclosure is described below.

### <Support>

The support of the electrophotographic photosensitive member is preferably a support having electroconductivity (electroconductive support). In addition, examples of the shape of the support include a cylindrical shape, a belt shape, and a sheet shape. Of those, a cylindrical support is preferred. In addition, the surface of the support may be subjected to, for example, electrochemical treatment such as anodization, blast treatment, or cutting treatment.

A metal, a resin, glass, or the like is preferred as a material for the support. Examples of the metal include aluminum, iron, nickel, copper, gold, stainless steel, and alloys thereof. Of those, an aluminum-made support using aluminum is preferred. In addition, electroconductivity is preferably imparted to the resin or the glass through treatment involving, for example, mixing or coating the resin or the glass with an electroconductive material.

### <Electroconductive Layer>

An electroconductive layer may be arranged on the support, though the electroconductive layer is not illustrated in FIG. 1. When the electroconductive layer is arranged, flaws and unevenness in the surface of the support can be concealed, and reflection of light on the surface of the support can be controlled.

The electroconductive layer preferably contains electroconductive particles and a resin. A material for the electroconductive particles is, for example, a metal oxide, a metal, or carbon black. Examples of the metal oxide include zinc oxide, aluminum oxide, indium oxide, silicon oxide, zirconium oxide, tin oxide, titanium oxide, strontium titanate, magnesium oxide, antimony oxide, and bismuth oxide. Examples of the metal include aluminum, nickel, iron, nichrome, copper, zinc, and silver. Of those, metal oxide particles are preferably used as the electroconductive particles, and in particular, titanium oxide particles, tin oxide particles, and zinc oxide particles are more preferably used.

When the metal oxide particles are used as the electroconductive particles, the surface of each of the metal oxide particles may be treated with a silane coupling agent or the like, or the metal oxide particles may each be doped with an element, such as phosphorus or aluminum, or an oxide thereof.

In addition, the electroconductive particles may each have a laminated configuration including a core particle and a coating layer coating the particle. Examples of the core particle include titanium oxide particles, barium sulfate particles, and zinc oxide particles. A material for the coating layer is, for example, metal oxide particles such as tin oxide.

In addition, when the metal oxide particles are used as the electroconductive particles, their volume-average particle diameter is preferably 1 nm or more and 500 nm or less, more preferably 3 nm or more and 400 nm or less.

Examples of the resin include a polyester resin, a polycarbonate resin, a polyvinyl acetal resin, an acrylic resin, a silicone resin, an epoxy resin, a melamine resin, a polyurethane resin, a phenol resin, and an alkyd resin.

In addition, the electroconductive layer may further contain, for example, a silicone oil, resin particles, or a concealing agent such as titanium oxide.

The electroconductive layer may be formed by preparing a coating liquid for an electroconductive layer containing the above-mentioned materials and a solvent, forming a coating film thereof on the support, and drying the coating film. Examples of the solvent to be used in the coating liquid for an electroconductive layer include an alcohol-based solvent, a sulfoxide-based solvent, a ketone-based solvent, an ether-based solvent, an ester-based solvent, and an aromatic hydrocarbon-based solvent. A dispersion method for dispersing the electroconductive particles in the coating liquid for an electroconductive layer is, for example, a method including using a paint shaker, a sand mill, a ball mill, or a liquid collision-type high-speed disperser.

The thickness of the electroconductive layer is preferably 1 μm or more and 50 μm or less, particularly preferably 3 μm or more and 40 μm or less.

### <Undercoat Layer>

In the present disclosure, the undercoat layer may be arranged on the support or the electroconductive layer. The arrangement of the undercoat layer enables an improvement in adhesive function between layers to impart a charge injection-inhibiting function. The undercoat layer preferably contains a resin. In addition, the undercoat layer may be formed as a cured film by polymerizing a composition containing a monomer having a polymerizable functional group.

Examples of the resin include a polyester resin, a polycarbonate resin, a polyvinyl acetal resin, an acrylic resin, an epoxy resin, a melamine resin, a polyurethane resin, a phenol resin, a polyvinyl phenol resin, an alkyd resin, a polyvinyl alcohol resin, a polyethylene oxide resin, a polypropylene oxide resin, a polyamide resin, a polyamic acid resin, a polyimide resin, a polyamide imide resin, and a cellulose resin.

Examples of the polymerizable functional group of the monomer having a polymerizable functional group include an isocyanate group, a blocked isocyanate group, a methylol group, an alkylated methylol group, an epoxy group, a metal alkoxide group, a hydroxy group, an amino group, a carboxy group, a thiol group, a carboxylic acid anhydride group, and a carbon-carbon double bond group.

In addition, the undercoat layer may further contain an electron-transporting substance, metal oxide particles, metal particles, an electroconductive polymer, and the like for the purpose of improving electric characteristics. Of those, an electron-transporting substance and metal oxide particles are preferably used.

Examples of the electron-transporting substance include a quinone compound, an imide compound, a benzimidazole compound, a cyclopentadienylidene compound, a fluorenone compound, a xanthone compound, a benzophenone compound, a cyanovinyl compound, a halogenated aryl compound, a silole compound, and a boron-containing compound. An electron-transporting substance having a polymerizable functional group may be used as the electron-transporting substance and copolymerized with the above-mentioned monomer having a polymerizable functional group to form the undercoat layer as a cured film.

Examples of the metal oxide particles include particles of indium tin oxide, tin oxide, indium oxide, titanium oxide, strontium titanate, zinc oxide, and aluminum oxide. Particles of silicon dioxide may also be used. Examples of the metal particles include particles of gold, silver, and aluminum.

The metal oxide particles to be incorporated into the undercoat layer may be subjected to surface treatment with a surface treatment agent such as a silane coupling agent before use. A general method is used as a method of subjecting the metal oxide particles to the surface treatment. Examples thereof include a dry method and a wet method.

The dry method involves, while stirring the metal oxide particles in a mixer capable of high-speed stirring such as a Henschel mixer, adding an alcoholic aqueous solution, organic solvent solution, or aqueous solution containing the surface treatment agent, uniformly dispersing the mixture, and then drying the dispersion.

The wet method involves stirring the metal oxide particles and the surface treatment agent in a solvent, or dispersing the metal oxide particles and the surface treatment agent in a solvent with a sand mill or the like using glass beads or the like. After the dispersion, the solvent is removed by filtration or evaporation under reduced pressure. After the removal of the solvent, it is preferred to further perform baking at 100° C. or more.

The undercoat layer may further contain an additive, and for example, may contain a known material, such as: metal particles such as aluminum particles; electroconductive particles such as carbon black; a charge-transporting substance; a metal chelate compound; or an organometallic compound.

The undercoat layer may be formed by preparing a coating liquid for an undercoat layer containing the above-mentioned materials and a solvent, forming a coating film thereof on the support or the electroconductive layer, and drying and/or curing the coating film.

Examples of the solvent to be used in the coating liquid for an undercoat layer include organic solvents, such as an alcohol-based solvent, a sulfoxide-based solvent, a ketone-based solvent, an ether-based solvent, an ester-based solvent, an aliphatic halogenated hydrocarbon-based solvent, and an aromatic compound-based solvent. In the present disclosure, an alcohol-based solvent and a ketone-based solvent are preferably used.

A dispersion method for preparing the coating liquid for an undercoat layer is, for example, a method including using a homogenizer, an ultrasonic disperser, a ball mill, a sand mill, a roll mill, a vibration mill, an attritor, or a liquid collision-type high-speed disperser.

The thickness of the undercoat layer is preferably 0.1 μm or more, more preferably 0.2 μm or more, particularly preferably 0.3 μm or more. In addition, the thickness of the undercoat layer is preferably 50 μm or less, more preferably 40 μm or less, more preferably 30 μm or less, more preferably 10 μm or less, particularly preferably 5 μm or less.

### <Photosensitive Layer>

The photosensitive layers of the electrophotographic photosensitive member are mainly classified into (1) a laminate-type photosensitive layer and (2) a monolayer-type photosensitive layer. (1) The laminate-type photosensitive layer is a photosensitive layer including a charge-generating layer containing a charge-generating substance and a charge-transporting layer containing a charge-transporting substance. (2) The monolayer-type photosensitive layer is a photosensitive layer containing both of a charge-generating substance and a charge-transporting substance.

### (1) Laminate-Type Photosensitive Layer

The laminate-type photosensitive layer include the charge-generating layer and the charge-transporting layer.

### (1-1) Charge-Generating Layer

The charge-generating layer preferably contains the charge-generating substance and a resin.

Examples of the charge-generating substance include azo pigments, perylene pigments, polycyclic quinone pigments, indigo pigments, and phthalocyanine pigments. Of those, azo pigments and phthalocyanine pigments are preferred. Of the phthalocyanine pigments, an oxytitanium phthalocyanine pigment, a chlorogallium phthalocyanine pigment, and a hydroxygallium phthalocyanine pigment are preferred. The content of the charge-generating substance in the charge-generating layer is preferably 40 mass % or more and 85 mass % or less, more preferably 60 mass % or more and 80 mass % or less with respect to the total mass of the charge-generating layer.

Examples of the resin include a polyester resin, a polycarbonate resin, a polyvinyl acetal resin, a polyvinyl butyral resin, an acrylic resin, a silicone resin, an epoxy resin, a melamine resin, a polyurethane resin, a phenol resin, a polyvinyl alcohol resin, a cellulose resin, a polystyrene resin, a polyvinyl acetate resin, and a polyvinyl chloride resin. Of those, a polyvinyl butyral resin is more preferred.

In addition, the charge-generating layer may further contain an additive, such as an antioxidant or a UV absorber. Specific examples thereof include a hindered phenol compound, a hindered amine compound, a sulfur compound, a phosphorus compound, and a benzophenone compound.

The charge-generating layer may be formed by preparing a coating liquid for a charge-generating layer containing the above-mentioned materials and a solvent, forming a coating film thereof on the undercoat layer, and drying the coating film. Examples of the solvent to be used in the coating liquid include an alcohol-based solvent, a sulfoxide-based solvent, a ketone-based solvent, an ether-based solvent, an ester-based solvent, and an aromatic hydrocarbon-based solvent.

The thickness of the charge-generating layer is preferably 0.1 μm or more and 1 μm or less, more preferably 0.15 μm or more and 0.4 μm or less.

### (1-2) Charge-Transporting Layer

The charge-transporting layer preferably contains the charge-transporting substance and a binder material.

Examples of the charge-transporting substance include a polycyclic aromatic compound, a heterocyclic compound, a hydrazone compound, a styryl compound, an enamine compound, a triarylamine compound, and a resin having a group derived from each of those substances. Of those, a triarylamine compound is preferred.

The content of the charge-transporting substance in the charge-transporting layer is preferably 25 mass % or more and 70 mass % or less, more preferably 30 mass % or more and 55 mass % or less with respect to the total mass of the charge-transporting layer.

A thermoplastic resin (hereinafter also referred to as “resin”) is used as the binder material. Examples of the thermoplastic resin include a polyester resin, a polycarbonate resin, an acrylic resin, and a polystyrene resin. Of those, a polycarbonate resin and a polyester resin are preferred. The polyester resin is particularly preferably a polyarylate resin.

A content ratio (mass ratio) between the charge-transporting substance and the resin is preferably from 4:10 to 20:10, more preferably from 5:10 to 12:10.

In addition, the charge-transporting layer may contain an additive, such as an antioxidant, a UV absorber, a plasticizer, or a leveling agent. Specific examples thereof include a hindered phenol compound, a hindered amine compound, a sulfur compound, a phosphorus compound, a benzophenone compound, a siloxane-modified resin, a silicone oil, polystyrene resin particles, polyethylene resin particles, and boron nitride particles.

The charge-transporting layer may be formed by preparing a coating liquid for a charge-transporting layer containing the above-mentioned materials and a solvent, forming a coating film thereof on the charge-generating layer, and drying the coating film. Examples of the solvent to be used in the coating liquid include an alcohol-based solvent, a ketone-based solvent, an ether-based solvent, an ester-based solvent, and an aromatic hydrocarbon-based solvent. Of those solvents, an ether-based solvent or an aromatic hydrocarbon-based solvent is preferred.

The thickness of the charge-transporting layer is preferably 5 μm or more and 50 μm or less, more preferably 8 μm or more and 40 μm or less, particularly preferably 10 μm or more and 30 μm or less.

### (2) Monolayer-Type Photosensitive Layer

The monolayer-type photosensitive layer may be formed by preparing a coating liquid for a photosensitive layer containing the charge-generating substance, the charge-transporting substance, a resin, and a solvent, forming a coating film thereof on the undercoat layer, and drying the coating film. Examples of the charge-generating substance, the charge-transporting substance, and the resin are the same as those of the materials in the section “(1) Laminate-type Photosensitive Layer.”

### <Protective Layer>

In the present disclosure, the protective layer serves as the surface layer of the electrophotographic photosensitive member. In this case, the protective layer contains a polymerized product of at least one kind of polymerizable charge-transporting compound selected from the group consisting of compounds represented by the formulae (CT-1) and (CT-2), and in dynamic viscoelasticity measurement, the protective layer satisfies the relationship of storage elastic modulus (G′)>loss elastic modulus (G″) at normal temperature, and has a peak top of Tan δ in a temperature range of 85° C. or more and 100° C. or less. In the present disclosure, the protective layer may further contain a polymerized product of a composition containing a thiol compound represented by the formula (T-1). The polymerizable charge-transporting compound to be used in the surface layer of the electrophotographic photosensitive member is more preferably the compound represented by the formula (CT-1).

The protective layer may contain an additive, such as an antioxidant, a UV absorber, a plasticizer, or a leveling agent. Specific examples thereof include a hindered phenol compound, a hindered amine compound, a phosphorus compound, a benzophenone compound, a siloxane-modified resin, a silicone oil, and a silicone compound. An example of the silicone compound may be a silicone compound having an acryloyloxy group or a methacryloyloxy group such as a silicone-modified acrylic resin. At this time, a silicone compound having a methacryloyloxy group is more preferred.

The protective layer may be formed by preparing a coating liquid for a protective layer containing the above-mentioned materials and a solvent, forming a coating film thereof on the photosensitive layer, and drying and/or curing the coating film. Examples of the solvent to be used in the coating liquid include an alcohol-based solvent, a ketone-based solvent, an ether-based solvent, a sulfoxide-based solvent, an ester-based solvent, and an aromatic hydrocarbon-based solvent.

The thickness of the protective layer is preferably 0.5 μm or more and 10 μm or less, more preferably 1 μm or more and 7 μm or less.

### <Surface Processing of Electrophotographic Photosensitive Member>

In the present disclosure, surface processing of the electrophotographic photosensitive member may be performed. When the surface processing is performed, the behavior of a cleaning unit (cleaning blade) to be brought into contact with the electrophotographic photosensitive member can be further stabilized. Examples of a method for the surface processing include a method including bringing a mold having a convex portion into pressure contact with the surface of the electrophotographic photosensitive member to perform shape transfer, a method including imparting concave and convex shapes by mechanical polishing, and a method including causing powder to collide with the surface of the electrophotographic photosensitive member to roughen the surface. When the concave portion or the convex portion is formed on the surface layer of the electrophotographic photosensitive member as described above, the behavior of the cleaning unit to be brought into contact with the electrophotographic photosensitive member can be further stabilized.

The concave portion or the convex portion may be formed on the entire surface of the electrophotographic photosensitive member, or may be formed on part of the surface of the electrophotographic photosensitive member. When the concave portion or the convex portion is formed on part of the surface of the electrophotographic photosensitive member, it is preferred that the concave portion or the convex portion be formed at least on the entire contact region with the cleaning unit (cleaning blade). When the concave portion is formed, the concave portion may be formed on the surface of the electrophotographic photosensitive member by bringing a mold having a convex portion corresponding to the concave portion into pressure contact with the surface of the electrophotographic photosensitive member to perform shape transfer.

### <Polishing Apparatus>

FIG. 2 is an illustration of an example of a polishing apparatus for the electrophotographic photosensitive member of the present disclosure.

FIG. 2 is an illustration of an apparatus for polishing a cylindrical electrophotographic photosensitive member using a polishing sheet. In FIG. 2, a polishing sheet 2-1 is wound around a hollow shaft 2-6, and a motor (not shown) is arranged so that tension is applied to the polishing sheet 2-1 in a direction opposite to a direction in which the polishing sheet 2-1 is fed to the shaft 2-6. The polishing sheet 2-1 is fed in the direction indicated by the arrow, and passes through a backup roller 2-3 via guide rollers 2-2a and 2-2b, and the polishing sheet 2-1 after polishing is wound around a winding unit 2-5 by a motor (not shown) via guide rollers 2-2c and 2-2d. The polishing is performed by constantly bringing the polishing sheet 2-1 into pressure contact with an object (electrophotographic photosensitive member before polishing) 2-4 to be processed. The polishing sheet 2-1 often has an insulating property, and hence it is preferred to use a member that is grounded or has electroconductivity at a portion where the polishing sheet 2-1 is brought into contact.

The feed speed of the polishing sheet 2-1 preferably falls within the range of from 10 mm/min to 1,000 mm/min. When a feed amount is small, adhesion of a binder resin to the surface of the polishing sheet 2-1 may occur, and as a result, deep scratches may be formed on the surface of the object 2-4 to be processed.

The object 2-4 to be processed is placed at a position facing the backup roller 2-3 via the polishing sheet 2-1. The backup roller 2-3 is preferably an elastic body from the viewpoint of improving the uniformity of the surface roughness of the object 2-4 to be processed. At this time, the object 2-4 to be processed and the backup roller 2-3 are pressed against each other via the polishing sheet 2-1 at a desired set value for a predetermined time, and the surface of the object 2-4 to be processed is polished. The rotation direction of the object 2-4 to be processed may be the same as the direction in which the polishing sheet 2-1 is fed, or may be opposite thereto. In addition, the rotation direction may be changed during polishing.

The pressing pressure of the backup roller 2-3 against the object 2-4 to be processed is preferably from 0.005 N/m² to 15 N/m² depending on the hardness of the backup roller 2-3 and the polishing time. The surface roughness of the electrophotographic photosensitive member may be adjusted by appropriately selecting the feed speed of the polishing sheet 2-1, the pressing pressure of the backup roller 2-3, the abrasive grain type of the polishing sheet, the thickness of the binder resin of the polishing sheet, the thickness of the substrate, or the like.

### <Measurement of Maximum Height Rmax in JIS B 0601:1982>

The surface roughness of the electrophotographic photosensitive member may be measured by a known unit. Examples thereof include a surface roughness meter such as Surface Roughness Tester Surfcorder SE3500 manufactured by Kosaka Laboratory Ltd., and a microscope capable of acquiring a three-dimensional shape, such as Ultra-Deep Shape Measurement Microscopes VK-8550 and VK-9000 manufactured by Keyence Corporation.

### <Process Cartridge and Electrophotographic Apparatus>

The electrophotographic photosensitive member of the present disclosure may be one constituent for a process cartridge or an electrophotographic apparatus. The process cartridge has features of integrally supporting the electrophotographic photosensitive member described in the foregoing, and at least one unit selected from the group consisting of: a charging unit; a developing unit; and a cleaning unit, and being detachably attachable to the main body of an electrophotographic apparatus. In addition, the electrophotographic apparatus has a feature of including the electrophotographic photosensitive member described in the foregoing and at least one unit selected from the group consisting of: a charging unit; an exposing unit; a developing unit; and a transfer unit.

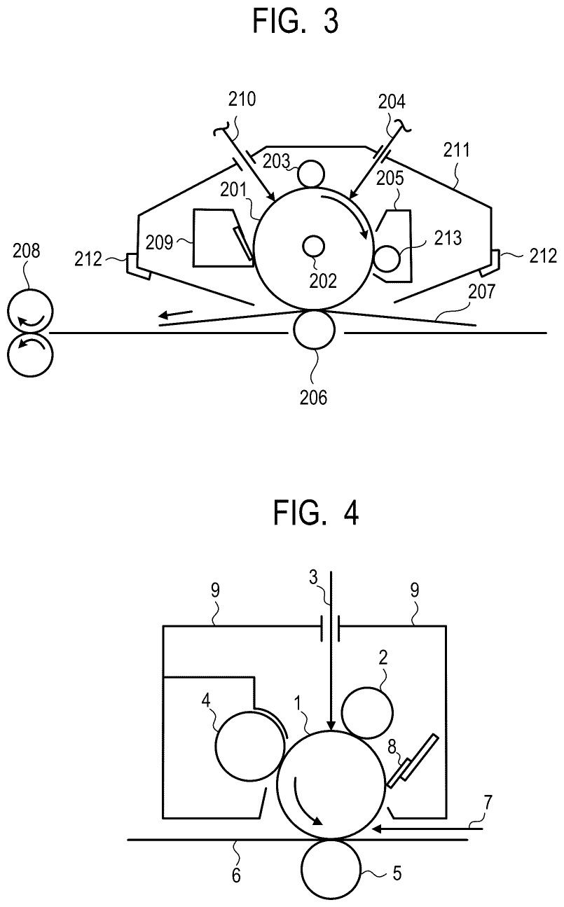

An example of the schematic configuration of an electrophotographic apparatus including a process cartridge including the electrophotographic photosensitive member of the present disclosure is illustrated in FIG. 3.

An electrophotographic photosensitive member 201 of a cylindrical shape (drum shape) is rotationally driven about a shaft 202 in a direction indicated by the arrow at a predetermined peripheral speed (process speed). The surface of the electrophotographic photosensitive member 201 is charged to a predetermined positive or negative potential by a charging unit 203 in the rotational process. In FIG. 3, a roller charging system based on a roller-type charging member is illustrated, but a charging system, such as a corona charging system, a proximity charging system, or an injection charging system, may be adopted. The charged surface of the electrophotographic photosensitive member 201 is irradiated with exposure light 204 from an exposing unit (not shown), and thus an electrostatic latent image corresponding to target image information is formed thereon. The exposure light 204 is light whose intensity has been modulated in correspondence with a time-series electric digital image signal of the target image information, and is emitted, for example, from an image exposing unit, such as slit exposure or laser beam scanning exposure.

The electrostatic latent image formed on the surface of the electrophotographic photosensitive member 201 is developed (normal development or reversal development) with toner on a developing member 213 stored in a developing unit 205 to form a toner image on the surface of the electrophotographic photosensitive member 201. The toner image formed on the surface of the electrophotographic photosensitive member 201 is transferred by a transfer unit 206 onto a transfer material 207. At this time, a bias voltage opposite in polarity to a charge that the toner possesses is applied from a bias power source (not shown) to the transfer unit 206. In addition, when the transfer material 207 is paper, the transfer material 207 is taken out of a sheet feeding portion (not shown) and fed to a space between the electrophotographic photosensitive member 201 and the transfer unit 206 in synchronization with the rotation of the electrophotographic photosensitive member 201.

The transfer material 207 onto which the toner image has been transferred from the electrophotographic photosensitive member 201 is separated from the surface of the electrophotographic photosensitive member 201, is conveyed to a fixing unit 208, and is subjected to treatment for fixing the toner image to be printed out as an image-formed product (a print or a copy) to the outside of the electrophotographic apparatus. The electrophotographic apparatus may include a cleaning unit 209 for removing a deposit such as the toner remaining on the surface of the electrophotographic photosensitive member 201 after the transfer. In addition, a so-called cleaner-less system that removes the deposit with the developing unit or the like without separate arrangement of the cleaning unit may be used.

A plurality of constituents selected from the electrophotographic photosensitive member 201, the charging unit 203, the developing unit 205, the cleaning unit 209, and the like may be stored in a container and integrally supported to form a process cartridge. In addition, the process cartridge may be detachably attachable to the main body of the electrophotographic apparatus. For example, at least one selected from the charging unit 203, the developing unit 205, and the cleaning unit 209 may be integrally supported with the electrophotographic photosensitive member 201 to form a cartridge. The cartridge may be used as a process cartridge 211 to be detachably attachable onto the main body of the electrophotographic apparatus with a guiding unit 212 such as a rail of the main body of the electrophotographic apparatus. The electrophotographic apparatus may include a charge-eliminating mechanism for subjecting the surface of the electrophotographic photosensitive member 201 to charge-eliminating treatment with pre-exposure light 210 from a pre-exposing unit (not shown). In addition, the guiding unit 212 such as the rail may be arranged for detachably attaching the process cartridge 211 onto the main body of the electrophotographic apparatus. The electrophotographic apparatus of the present disclosure may include the electrophotographic photosensitive member 201, and the charging unit 203, the exposing unit, the developing unit 205, and the transfer unit 206.

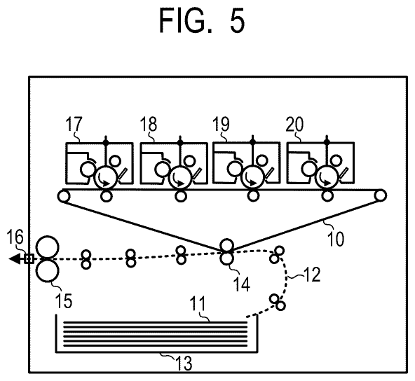

In addition, an example of the schematic configuration of a process cartridge including the electrophotographic photosensitive member of the present disclosure is illustrated in FIG. 4, and an example of the schematic configuration of an electrophotographic apparatus including the process cartridge of FIG. 4 is illustrated in FIG. 5.

In FIG. 4, a cylindrical electrophotographic photosensitive member 1 is driven to rotate at a predetermined peripheral speed in a direction indicated by the arrow. The circumferential surface of the electrophotographic photosensitive member 1 that is driven to rotate is uniformly charged to a positive or negative predetermined potential by a charging unit 2. Then, the charged circumferential surface of the electrophotographic photosensitive member 1 receives exposure light (image exposure light) 3 emitted from an exposing unit (not shown), such as slit exposure light or laser beam scanning exposure light. Thus, an electrostatic latent image corresponding to an intended image is sequentially formed on the circumferential surface of the electrophotographic photosensitive member 1. Any one of a voltage in which an AC component is superimposed on a DC component or a voltage formed of only a DC component may be used as a voltage to be applied to the charging unit (e.g., a charging roller) 2.

The electrostatic latent image formed on the circumferential surface of the electrophotographic photosensitive member 1 is developed with a toner in a developer of a developing unit 4 to form a toner image. Then, the toner image formed and borne on the circumferential surface of the electrophotographic photosensitive member 1 is sequentially transferred onto a transfer material (e.g., paper or an intermediate transfer member) 6 with a transfer bias from a transfer unit (e.g., a transfer roller) 5. The transfer material 6 is fed in synchronization with the rotation of the electrophotographic photosensitive member 1.

The surface of the electrophotographic photosensitive member 1 after the transfer of the toner image is subjected to charge-eliminating treatment with pre-exposure light 7 from a pre-exposing unit (not shown). After that, the surface is cleaned by removal of a transfer residual toner with a cleaning unit 8. Then, the electrophotographic photosensitive member 1 is repeatedly used in image formation. The treatment by the pre-exposing unit may be performed before or after a cleaning step, and the pre-exposing unit is not necessarily required.

The electrophotographic photosensitive member 1 may be mounted on an electrophotographic apparatus, such as a copying machine or a laser beam printer. In addition, a process cartridge 9 configured to integrally support a plurality of constituent elements, such as the electrophotographic photosensitive member 1, the charging unit 2, the developing unit 4, and the cleaning unit 8, accommodated in a container may be configured to be detachably attachable onto a main body of an electrophotographic apparatus. In FIG. 4, the electrophotographic photosensitive member 1, the charging unit 2, the developing unit 4, and the cleaning unit 8 are integrally supported to form the process cartridge 9 that is detachably attachable onto a main body of an electrophotographic apparatus.

Next, an electrophotographic apparatus including the electrophotographic photosensitive member of the present disclosure is described.

An example of the configuration of the electrophotographic apparatus of the present disclosure is illustrated in FIG. 5. A process cartridge 17 for yellow color, a process cartridge 18 for magenta color, a process cartridge 19 for cyan color, and a process cartridge 20 for black color, corresponding to yellow color, magenta color, cyan color, and black color, respectively, are arranged side by side along an intermediate transfer member 10. The diameter and constituent materials of the electrophotographic photosensitive member, a developer, a charging system, and other units are not necessarily required to be the same for each color.

When an image forming operation is started, a toner image of each color is sequentially superimposed on the intermediate transfer member 10 in accordance with the above-mentioned image forming process. In parallel, a transfer sheet 11 is sent from a sheet feeding tray 13 through a sheet feeding path 12 and fed to a secondary transfer unit 14 in synchronization with the timing of the rotation operation of the intermediate transfer member 10. A toner image on the intermediate transfer member 10 is transferred onto the transfer sheet 11 with a transfer bias from the secondary transfer unit 14. The toner image transferred onto the transfer sheet 11 is conveyed along the sheet feeding path 12 and fixed onto the transfer sheet by a fixing unit 15. Then, the transfer sheet 11 is delivered from a sheet delivery portion 16. The electrophotographic photosensitive member of the present disclosure may be used in a laser beam printer, an LED printer, a copying machine, a facsimile, a multifunctional peripheral thereof, and the like.

### EXAMPLES

The present disclosure is described in more detail below by way of Examples and Comparative Examples, but is not limited thereto. In the description of Examples below, the term “part(s)” means “part(s) by mass” unless otherwise stated.

### <Synthesis of Charge-Transporting Compound Represented by Formula (CT-1)>

The compound represented by the formula (CT-1) of the present disclosure may be synthesized by using, for example, a synthesis method described in Japanese Patent Laid-Open No. 2010-156835.

### Example 1-1

### (Support)

A product obtained by cutting a cylindrical aluminum cylinder (JIS-A3003, aluminum alloy, outer diameter: 30 mm, length: 357.5 mm, wall thickness: 0.7 mm) was used as a support (electroconductive support). The support was subjected to ultrasonic cleaning in a cleaning liquid obtained by incorporating a detergent (product name: CHEMICOL CT, manufactured by Tokiwa Chemical Industries Co., Ltd.) into pure water, and subsequently, the cleaning liquid was washed off. After that, the cleaned product was further subjected to ultrasonic cleaning in pure water to be subjected to degreasing treatment. The resultant was used as a support.

### (Undercoat Layer 1)

100 Parts of zinc oxide particles (specific surface area: 19 m²/g, powder resistance: 4.7×10⁶ Ω·cm) were stirred and mixed with 500 parts of toluene, and 0.8 part of a silane coupling agent (compound name: N-2-(aminoethyl)-3-aminopropylmethyldimethoxysilane, product name: KBM-602, manufactured by Shin-Etsu Chemical Co., Ltd.) was added to the mixture, followed by stirring for 6 hours. After that, toluene was evaporated under reduced pressure, and the residue was heated and dried at 130° C. for 6 hours to provide surface-treated zinc oxide particles A.

Subsequently, 15 parts of polyvinyl butyral (product name: S-LEC (trademark) BM-1, manufactured by Sekisui Chemical Co., Ltd.) serving as a polyol and 15 parts of a blocked isocyanate (product name: DURANATE (trademark) TPA-B80E, non-volatile content of 80% by mass, manufactured by Asahi Kasei Chemicals Corporation) were dissolved in a mixed solvent including 73.5 parts of methyl ethyl ketone and 73.5 parts of 1-butanol. To this solution, 80.8 parts of the surface-treated zinc oxide particles A and 0.81 part of 2,3,4-trihydroxybenzophenone (manufactured by Tokyo Chemical Industry Co., Ltd.) were added, and the materials were dispersed in a sand mill apparatus using glass beads each having a diameter of 0.8 mm for 3 hours in an atmosphere of 23±3° C.

After the dispersion treatment, 0.01 part of silicone oil (product name: SH28PA, manufactured by Dow Toray Co., Ltd. (formerly: Toray Dow Corning Silicone Co., Ltd.)) and 5.6 parts of crosslinked polymethyl methacrylate (PMMA) particles (product name: TECHPOLYMER (trademark) SSX-103, manufactured by Sekisui Kasei Co., Ltd., average primary particle diameter: 3 μm) were added to the resultant, and the mixture was stirred to prepare a coating liquid for an undercoat layer. The resultant coating liquid for an undercoat layer was applied onto the above-mentioned support by dip coating to form a coating film, and the coating film was dried at 160° C. for 30 minutes to form an undercoat layer 1 having a thickness of 18 μm.

### (Charge-Generating Layer)

4 Parts of a hydroxygallium phthalocyanine crystal (charge-generating substance) in a crystal form having strong peaks at Bragg angles 2θ±0.2° of 7.4° and 28.1° in CuKα characteristic X-ray diffraction, and 0.04 part of a compound represented by the formula (E) were added to a liquid obtained by dissolving 2 parts of polyvinyl butyral (product name: S-LEC BX-1, manufactured by Sekisui Chemical Co., Ltd.) in 100 parts of cyclohexanone. After that, the mixture was subjected to dispersion treatment with a sand mill using glass beads each having a diameter of 1 mm for 1 hour in an atmosphere of 23±3° C. After the dispersion treatment, 100 parts of ethyl acetate was added to prepare a coating liquid for a charge-generating layer.

The coating liquid for a charge-generating layer was applied onto the above-mentioned undercoat layer 1 by dip coating, and the resultant coating film was dried at 90° C. for 10 minutes to form a charge-generating layer having a thickness of 0.15 μm.

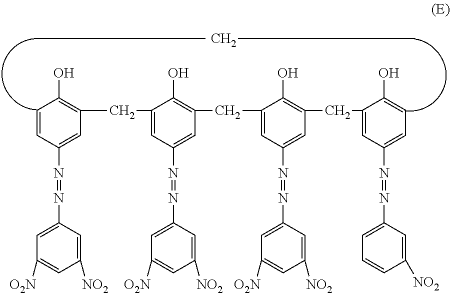

### (Charge-Transporting Layer)

60 Parts of a compound represented by the formula (F), 30 parts of a compound represented by the formula (G), 10 parts of a compound represented by the formula (H), 100 parts of a bisphenol Z-type polycarbonate resin (product name: Iupilon Z400, manufactured by Mitsubishi Engineering-Plastics Corporation), and 0.2 part of a polycarbonate having a structural unit represented by the formula (I) (viscosity average molecular weight Mv: 20,000) were dissolved in a mixed solvent including 272 parts of o-xylene, 256 parts of methyl benzoate, and 272 parts of dimethoxymethane to prepare a coating liquid for a charge-transporting layer.

The coating liquid for a charge-transporting layer was applied onto the above-mentioned charge-generating layer by dip coating to form a coating film, and the resultant coating film was dried at 115° C. for 50 minutes to form a charge-transporting layer having a thickness of 18 μm.

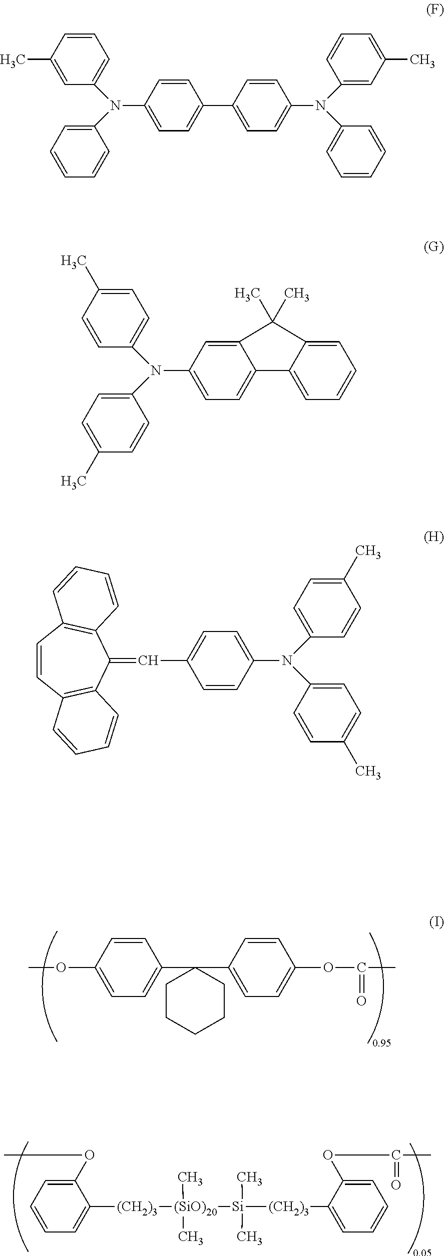

In the formula (I), 0.95 and 0.05 are molar ratios (copolymerization ratios) of the two structural units.

### (Protective Layer)

Next, 60 parts of a compound represented by the formula (CT1-1) serving as the compound represented by the formula (CT-1) was dissolved in 70 parts of n-propanol, and 70 parts of 1,1,2,2,3,3,4-heptafluorocyclopentane (product name: Zeorora (trademark) hyH, manufactured by Zeon Corporation) was further added to prepare a coating liquid for a protective layer. After that, the resultant was filtered with a polyflon filter (product name: PF-040, manufactured by Advantec Toyo Kaisha, Ltd.) to prepare a coating liquid for a protective layer.

The coating liquid for a protective layer was applied onto the charge-transporting layer by dip coating to form a coating film, and the resultant coating film was dried at 40° C. for 5 minutes. After the drying, the coating film was irradiated with an electron beam for 1.6 seconds under the conditions of an acceleration voltage of 70 kV and an absorbed dose of 15 kGy under a nitrogen atmosphere. After that, heating treatment was performed for 15 seconds under the condition that the temperature of the coating film became 135° C. under a nitrogen atmosphere. An oxygen concentration in a time period from the electron beam irradiation to the heating treatment for 15 seconds was 15 ppm. Next, natural cooling was performed in the air until the temperature of the coating film became 25° C. After that, heating treatment was performed for 1 hour under the condition that the temperature of the coating film became 105° C. to form a protective layer having a thickness of 5 μm. Thus, an electrophotographic photosensitive member including a support and a surface layer before surface polishing was produced. The results of the dynamic viscoelasticity measurement of the produced protective layer were as follows: storage elastic modulus (G′)>loss elastic modulus (G″) was satisfied at normal temperature; and the peak top temperature of Tan δ was 90° C.

### <Surface Processing of Electrophotographic Photosensitive Member>

(Polishing of Electrophotographic Photosensitive Member before Surface Polishing)

The surface of the electrophotographic photosensitive member before surface shape formation was polished. The polishing was performed with the polishing apparatus of FIG. 2 under the following conditions.
Feed speed of polishing sheet: 400 mm/min
Rotation speed of electrophotographic photosensitive member: 450 rpm
Indentation of electrophotographic photosensitive member into backup roller: 3.5 mm
Rotation directions of polishing sheet and electrophotographic photosensitive member: same

### Examples 1-2 to 1-5

Electrophotographic photosensitive members were produced in the same manner as in Example 1-1 except that, in the formation of the protective layer, the polymerizable charge-transporting compound represented by the formula (CT1-1) was changed to compounds represented by the formulae (CT1-2), (CT1-8), (CT1-7), and (CT1-6). The results of the dynamic viscoelasticity measurement of the produced protective layers were as follows: storage elastic modulus (G′)>loss elastic modulus (G″) was satisfied at normal temperature in all cases; and the peak top temperatures of Tan δ were 93° C., 96° C., 94° C., and 91° C., respectively.

### Examples 1-6 and 1-7

In the formation of the protective layer, the polymerizable charge-transporting compound represented by the formula (CT1-1) was changed to compounds represented by the formulae (CT1-10) and (CT1-12), and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT1-10) or (CT1-12) in the composition was represented by WCT, and the content of KarenzMT PE1 therein was represented by WT, the compounds were incorporated in amounts satisfying WT/WCT=0.06. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE was 0.001 or less in all cases. Electrophotographic photosensitive members were produced in the same manner as in Example 1-1 except for those conditions. The results of the dynamic viscoelasticity measurement of the produced protective layers were as follows: storage elastic modulus (G′)>loss elastic modulus (G″) was satisfied at normal temperature in all cases; and the peak top temperatures of Tan δ were 88° C. and 86° C., respectively.

### Examples 1-8 and 1-9

In the formation of the protective layer, the polymerizable charge-transporting compound represented by the formula (CT1-1) was changed to compounds represented by the formulae (CT1-4) and (CT1-11), and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, the following coating liquid for a protective layer was prepared: when the content of the polymerizable charge-transporting compound represented by the formula (CT1-4) or (CT1-11) in the composition was represented by WCT, and the content of KarenzMT PE1 therein was represented by WT, the compounds were incorporated in amounts satisfying WT/WCT-0.03. The coating liquid for a protective layer was applied onto the charge-transporting layer by dip coating to form a coating film, and the resultant coating film was dried at 40° C. for 5 minutes. After the drying, the coating film was irradiated with an electron beam for 1.6 seconds under the conditions of an acceleration voltage of 58 kV and an absorbed dose of 15 kGy under a nitrogen atmosphere. After that, heating treatment was performed for 15 seconds under the condition that the temperature of the coating film became 135° C. under a nitrogen atmosphere. An oxygen concentration in a time period from the electron beam irradiation to the heating treatment for 15 seconds was 15 ppm. Next, natural cooling was performed in the air until the temperature of the coating film became 25° C. After that, heating treatment was performed for 1 hour under the condition that the temperature of the coating film became 105° C. to form a protective layer having a thickness of 5 μm. At this time, as a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or more. Electrophotographic photosensitive members were produced in the same manner as in Example 1-1 except for those conditions. The results of the dynamic viscoelasticity measurement of the produced protective layers were as follows: storage elastic modulus (G′)>loss elastic modulus (G″) was satisfied at normal temperature in all cases; and the peak top temperatures of Tan δ were 86° C. and 89° C., respectively. [Examples 1-10 and 1-11]

In the formation of the protective layer, the polymerizable charge-transporting compound represented by the formula (CT1-1) was changed to compounds represented by the formulae (CT1-5) and (CT1-9), and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT1-5) or (CT1-9) in the composition was represented by WCT, and the content of KarenzMT PE1 therein was represented by WT, the compounds were incorporated in amounts satisfying WT/WCT-0.02. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. Electrophotographic photosensitive members were produced in the same manner as in Example 1-1 except for those conditions. The results of the dynamic viscoelasticity measurement of the produced protective layers were as follows: storage elastic modulus (G′)>loss elastic modulus (G″) was satisfied at normal temperature in all cases; and the peak top temperatures of Tan δ were 86° C. and 88° C., respectively.

### Example 1-12

In the formation of the protective layer, the polymerizable charge-transporting compound represented by the formula (CT1-1) was changed to a compound represented by the formula (CT1-3), and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT-3) in the composition was represented by WCT, and the content of KarenzMT PE1 therein was represented by WT, the compounds were incorporated in amounts satisfying WT/WCT-0.02, and 3 parts of a silicone-modified acrylic resin solution (product name: Symac (trademark) US-270, manufactured by Toagosei Co., Ltd.) was incorporated. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 85° C.

### Examples 1-13 and 1-14

In the formation of the protective layer, the polymerizable charge-transporting compound represented by the formula (CT1-1) was changed to a compound represented by the formula (CT2-1) or (CT2-2), and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT2-1) or (CT2-2) in the composition was represented by WCT, and the content of KarenzMT PE1 therein was represented by WT, the compounds were incorporated in amounts satisfying WT/WCT=0.02. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. Electrophotographic photosensitive members were produced in the same manner as in Example 1-1 except for those conditions. The results of the dynamic viscoelasticity measurement of the produced protective layers were as follows: storage elastic modulus (G′)>loss elastic modulus (G″) was satisfied at normal temperature in all cases; and the peak top temperatures of Tan δ were 95° C. and 100° C., respectively.

### Example 1-15

In the formation of the protective layer, a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT1-1) in the composition was represented by WCT, and the content of KarenzMT PE1 therein was represented by WT, the compounds were incorporated in amounts satisfying a WT/WCT value of 0.01. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 97° C.

### Example 1-16

In the formation of the protective layer, a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT1-1) in the composition was represented by WCT, and the content of KarenzMT PE1 therein was represented by WT, the compounds were incorporated in amounts satisfying a WT/WCT value of 0.07. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 88° C.

### Example 1-17

In the formation of the protective layer, the polymerizable charge-transporting compound represented by the formula (CT1-1) was changed to a compound represented by the formula (CT1-13) having 1 methacryloyl functional group, and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT1-13) in the composition was represented by WCT, and the content of KarenzMT PE1 therein was represented by WT, the compounds were incorporated in amounts satisfying WT/WCT=0.02. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 87° C.

### (CT1-13)

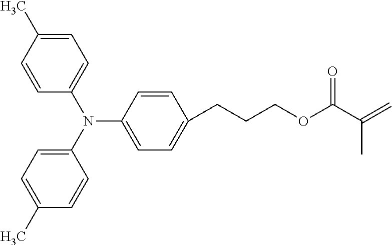 [SMILES: C=C(C)C(=O)OCCCc1ccc(N(c2ccc(C)cc2)c2ccc(C)cc2)cc1]

### Example 1-18

In the formation of the protective layer, the polymerizable charge-transporting compound represented by the formula (CT1-1) was changed to a compound represented by the formula (CT1-14) having 2 methacryloyl functional groups, and a polymerized product of KarenzMT PE1 (pentaerythritol tetrakis(3-mercaptobutyrate), manufactured by Resonac Corporation, a compound having 4 secondary thiol groups) serving as a composition containing a thiol compound was added. At this time, when the content of the polymerizable charge-transporting compound represented by the formula (CT1-14) in the composition was represented by WCT, and the content of KarenzMT PE1 therein was represented by WT, the compounds were incorporated in amounts satisfying WT/WCT-0.02. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT PE1, which was a thiol compound, was represented by AT, and the absorbance of a C═O stretching absorption band of an ester group in the range of from 1,735 cm⁻¹ to 1,750 cm⁻¹ derived from KarenzMT PE1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 95° C.

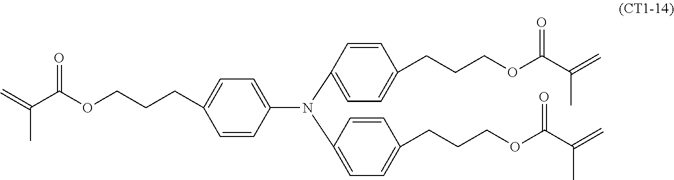 [SMILES: C=C(C)C(=O)OCCCc1ccc(N(c2ccc(CCCOC(=O)C(=C)C)cc2)c2ccc(CCCOC(=O)C(=C)C)cc2)cc1]

### Example 1-19

In the formation of the protective layer, a polymerized product of KarenzMT NR1 (1,3,5-tris(3-mercaptobutyryloxyethyl)-1,3,5-triazine-2,4,6(1H,3H,5H)-trione, manufactured by Resonac Corporation, a compound having 3 secondary thiol groups) serving as a composition containing a thiol compound was added. At this time, when the content of the polymerizable charge-transporting compound represented by the formula (CT1-1) in the composition was represented by WCT, and the content of KarenzMT NR1 therein was represented by WT, the compounds were incorporated in amounts satisfying a WT/WCT value of 0.02. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT NR1, which was a thiol compound, was represented by AT, and the absorbance of a C═O stretching absorption band of an ester group in the range of from 1,735 cm⁻¹ to 1,750 cm⁻¹ derived from KarenzMT NR1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 87° C.

### Example 1-20

In the formation of the protective layer, a polymerized product of KarenzMT BD1 (1,4-bis(3-mercaptobutyryloxy) butane, manufactured by Resonac Corporation, a compound having 2 secondary thiol groups) serving as a composition containing a thiol compound was added. At this time, when the content of the polymerizable charge-transporting compound represented by the formula (CT1-1) in the composition was represented by WCT, and the content of KarenzMT BD1 therein was represented by WT, the compounds were incorporated in amounts satisfying a WT/WCT value of 0.02. As a result of the determination of the residual SH amount of the protective layer by an infrared absorption spectrum, when the absorbance of a S—H stretching absorption band in the range of from 2,550 cm⁻¹ to 2,600 cm⁻¹ derived from KarenzMT BD1, which was a thiol compound, was represented by AT, and the absorbance of a C═O stretching absorption band of an ester group in the range of from 1,735 cm⁻¹ to 1,750 cm⁻¹ derived from KarenzMT BD1 was represented by AE, it was recognized that AT/AE became 0.001 or less. An electrophotographic photosensitive member was produced in the same manner as in Example 1-1 except for those conditions. The results of the dynamic viscoelasticity measurement of the produced protective layer were as follows: storage elastic modulus (G′)>loss elastic modulus (G″) was satisfied at normal temperature; and the peak top temperature of Tan δ was 89° C.

### Comparative Example 1-1

An electrophotographic photosensitive member was produced in the same manner as in Example 1-1 except that, in the formation of the protective layer, the compound represented by the formula (CT1-1) was changed to a compound represented by the formula (CT1-15) in which the functional group was an acrylic group. The results of the dynamic viscoelasticity measurement of the produced protective layer were as follows: storage elastic modulus (G′)>loss elastic modulus (G″) was satisfied at normal temperature; and the peak top temperature of Tan δ was 85° C.

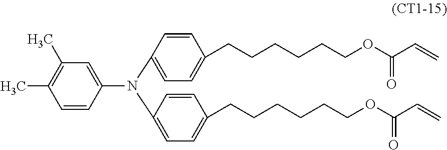 [SMILES: C=CC(=O)OCCCCCCc1ccc(N(c2ccc(CCCCCCOC(=O)C=C)cc2)c2ccc(C)c(C)c2)cc1]

### Comparative Example 1-2

In the formation of the protective layer, the coating liquid for a protective layer was applied onto the charge-transporting layer by dip coating to form a coating film, and the resultant coating film was dried at 40° C. for 5 minutes. After the drying, the coating film was irradiated with an electron beam for 1.6 seconds under the conditions of an acceleration voltage of 70 kV and an absorbed dose of 15 kGy under a nitrogen atmosphere. After that, heating treatment was performed for 15 seconds under the condition that the temperature of the coating film became 80° C. under a nitrogen atmosphere. An oxygen concentration in a time period from the electron beam irradiation to the heating treatment for 15 seconds was 15 ppm. Next, natural cooling was performed in the air until the temperature of the coating film became 25° C. After that, heating treatment was performed for 1 hour under the condition that the temperature of the coating film became 105° C. to form a protective layer having a thickness of 5 μm. At this time, the protective layer was produced so that the dynamic viscoelasticity was adjusted to satisfy storage elastic modulus (G′)<loss elastic modulus (G″) at normal temperature. The peak top temperature of Tan δ was 60° C.

An electrophotographic photosensitive member was produced in the same manner as in Example 1-1 except that the relationship between the storage elastic modulus G′ and the loss elastic modulus G″ in the dynamic viscoelasticity measurement was changed to G′<G″ at normal temperature.

### Comparative Example 1-3

An electrophotographic photosensitive member was produced in the same manner as in Example 1-12 except that KarenzMT PE1 was not used in the formation of the protective layer. The results of the dynamic viscoelasticity measurement of the produced protective layer were as follows: storage elastic modulus (G′)>loss elastic modulus (G″) was satisfied at normal temperature; and the peak top temperature of Tan δ was 125° C.

### Comparative Example 1-4

An electrophotographic photosensitive member was produced in the same manner as in Example 1-12 except that the content of the polymerizable charge-transporting compound represented by the formula (CT1-1) and the content of KarenzMT PE1 were changed so that the WT/WCT value became 0.09 in the formation of the protective layer. The results of the dynamic viscoelasticity measurement of the produced protective layer were as follows: storage elastic modulus (G′)>loss elastic modulus (G″) was satisfied at normal temperature; and the peak top temperature of Tan δ was 75° C.

### Examples 2-1 to 2-20 and Comparative Examples 2-1 to 2-4

Electrophotographic photosensitive members of Examples 2-1 to 2-20 and Comparative Examples 2-1 to 2-4 were produced in the same manner as in the production of the electrophotographic photosensitive members of Examples 1-1 to 1-20 and Comparative Examples 1-1 to 1-4 except that the undercoat layer 1 was changed to the following undercoat layer 2.

### (Undercoat Layer 2)

The undercoat layer 2 includes the following two layers: an electroconductive layer; and a nylon resin layer. First, the electroconductive layer was prepared as described below. 60 Parts by mass of TiO₂ particles coated with oxygen-deficient SnO₂ serving as electroconductive particles, 36.5 parts by mass of a phenol resin serving as a binder resin, and 20 parts by mass of methoxypropanol serving as a solvent were dispersed for 1 hour in a horizontal sand mill disperser using glass beads each having a diameter of 1 mm to prepare a dispersion liquid. In this case, the TiO₂ particles coated with oxygen-deficient SnO₂ as the electroconductive particles have a powder resistivity of 100 Ω·cm and a SnO₂ coverage (mass ratio) of 40%. In addition, the phenol resin is available under the product name “Plyophen J-325” from DIC Corporation, and has a resin solid content of 60%, 18 Kilograms of glass beads were internally added to the horizontal sand mill, and the rotation speed of a dispersion disc during dispersion was set to 900 rpm.

The average particle diameter of the TiO₂ particles coated with oxygen-deficient SnO₂ in the dispersion liquid was 0.36 μm. 1.5 Parts by mass of silicone resin particles serving as a surface roughening material and 0.001 part by mass of silicone oil serving as a leveling agent were added to the dispersion liquid, and the mixture was stirred to prepare a coating liquid for an electroconductive layer. In this case, the silicone resin particles are available under the product name “Tospearl (trademark) 120” from Momentive Performance Materials Japan LLC, and have an average particle diameter of 2 μm. In addition, the silicone oil is available under the product name “SH28PA” from Dow Toray Co., Ltd.

The coating liquid for an electroconductive layer was applied onto the support by dip coating, and the resultant was dried and thermally cured at 150° C. for 30 minutes to form an electroconductive layer having a thickness of 18 μm. The nylon resin layer was formed as described below. A solution prepared by dissolving 40 parts by mass of a methoxymethylated nylon 6 resin (product name: Toresin EF-30T, manufactured by Nagase ChemteX Corporation) in a mixed liquid including 400 parts by mass of methanol and 200 parts by mass of butanol was applied onto the above-mentioned electroconductive layer by dip coating, and the resultant was heated and dried for 30 minutes in a hot air dryer adjusted to 100° C. to cure the coating film of the solution, to thereby form a nylon resin layer having a thickness of 0.5 μm. Thus, the undercoat layer 2 was prepared.

With regard to Examples 1-1 to 1-20 and 2-1 to 2-20, and Comparative Examples 1-1 to 1-4 and 2-1 to 2-4, the WT/WCT value, viscoelastic characteristics, Tan δ peak top temperature, and residual SH amount of each protective layer are shown in Tables 1-1 and 1-2 below.

TABLE 1-1

| | | Polymerizable | Number of | Number of | |
|---|---|---|---|---|---|
| | | charge- | methacryloyl | acryloyl | |
| | | transporting | functional | functional | Thiol |
| | | compound | groups | groups | compound |
| Example 1-1 | Example 2-1 | CT1-1 | 3 | — | — |
| Example 1-2 | Example 2-2 | CT1-2 | 2 | — | — |
| Example 1-3 | Example 2-3 | CT1-8 | 2 | — | — |
| Example 1-4 | Example 2-4 | CT1-7 | 2 | — | — |
| Example 1-5 | Example 2-5 | CT1-6 | 2 | — | — |
| Example 1-6 | Example 2-6 | CT1-10 | 2 | — | KarenzMT PE1 |
| Example 1-7 | Example 2-7 | CT1-12 | 2 | — | KarenzMT PE1 |
| Example 1-8 | Example 2-8 | CT1-4 | 2 | — | KarenzMT PE1 |
| Example 1-9 | Example 2-9 | CT1-11 | 2 | — | KarenzMT PE1 |
| Example 1-10 | Example 2-10 | CT1-5 | 2 | — | KarenzMT PE1 |
| Example 1-11 | Example 2-11 | CT1-9 | 2 | — | KarenzMT PE1 |
| Example 1-12 | Example 2-12 | CT1-3 | 2 | — | KarenzMT PE1 |
| Example 1-13 | Example 2-13 | CT2-1 | 2 | — | KarenzMT PE1 |
| Example 1-14 | Example 2-14 | CT2-2 | 4 | — | KarenzMT PE1 |
| Example 1-15 | Example 2-15 | CT1-1 | 3 | — | KarenzMT PE1 |
| Example 1-16 | Example 2-16 | CT1-1 | 3 | — | KarenzMT PE1 |
| Example 1-17 | Example 2-17 | CT1-13 | 1 | — | KarenzMT PE1 |
| Example 1-18 | Example 2-18 | CT1-14 | 3 | — | KarenzMT PE1 |
| Example 1-19 | Example 2-19 | CT1-1 | 3 | — | KarenzMT NR1 |
| Example 1-20 | Example 2-20 | CT1-1 | 3 | — | KarenzMT BD1 |
| Comparative | Comparative | CT1-15 | — | 2 | — |
| Example 1-1 | Example 2-1 | | | | |
| Comparative | Comparative | CT1-1 | 3 | — | — |
| Example 1-2 | Example 2-2 | | | | |
| Comparative | Comparative | CT1-3 | 2 | — | — |
| Example 1-3 | Example 2-3 | | | | |
| Comparative | Comparative | CT1-3 | 2 | — | KarenzMT PE1 |
| Example 1-4 | Example 2-4 | | | | |

TABLE 1-2

| | | | | Tanδ peak top | | Residual SH |
|---|---|---|---|---|---|---|
| | | WT/WCT | Viscoelasticity | temperature | | amount |
| Example 1-1 | Example 2-1 | — | G′ > G″ | 90° | C. | <0.001 |
| Example 1-2 | Example 2-2 | — | G′ > G″ | 93° | C. | <0.001 |
| Example 1-3 | Example 2-3 | — | G′ > G″ | 96° | C. | <0.001 |
| Example 1-4 | Example 2-4 | — | G′ > G″ | 94° | C. | <0.001 |
| Example 1-5 | Example 2-5 | — | G′ > G″ | 91° | C. | <0.001 |
| Example 1-6 | Example 2-6 | 0.06 | G′ > G″ | 88° | C. | <0.001 |
| Example 1-7 | Example 2-7 | 0.06 | G′ > G″ | 86° | C. | <0.001 |
| Example 1-8 | Example 2-8 | 0.03 | G′ > G″ | 86° | C. | >0.001 |
| Example 1-9 | Example 2-9 | 0.03 | G′ > G″ | 89° | C. | >0.001 |
| Example 1-10 | Example 2-10 | 0.02 | G′ > G″ | 86° | C. | <0.001 |
| Example 1-11 | Example 2-11 | 0.02 | G′ > G″ | 88° | C. | <0.001 |
| Example 1-12 | Example 2-12 | 0.02 | G′ > G″ | 85° | C. | <0.001 |
| Example 1-13 | Example 2-13 | 0.02 | G′ > G″ | 95° | C. | <0.001 |
| Example 1-14 | Example 2-14 | 0.02 | G′ > G″ | 100° | C. | <0.001 |
| Example 1-15 | Example 2-15 | 0.01 | G′ > G″ | 97° | C. | <0.001 |
| Example 1-16 | Example 2-16 | 0.07 | G′ > G″ | 88° | C. | <0.001 |
| Example 1-17 | Example 2-17 | 0.02 | G′ > G″ | 87° | C. | <0.001 |
| Example 1-18 | Example 2-18 | 0.02 | G′ > G″ | 95° | C. | <0.001 |
| Example 1-19 | Example 2-19 | 0.02 | G′ > G″ | 87° | C. | <0.001 |
| Example 1-20 | Example 2-20 | 0.02 | G′ > G″ | 89° | C. | <0.001 |
| Comparative | Comparative | — | G′ > G″ | 85° | C. | <0.001 |
| Example 1-1 | Example 2-1 | | | | | |
| Comparative | Comparative | — | G′ < G″ | 60° | C. | <0.001 |
| Example 1-2 | Example 2-2 | | | | | |
| Comparative | Comparative | — | G′ > G″ | 125° | C. | <0.001 |
| Example 1-3 | Example 2-3 | | | | | |
| Comparative | Comparative | 0.09 | G′ > G″ | 75° | C. | <0.001 |
| Example 1-4 | Example 2-4 | | | | | |

### <Evaluation of Electrophotographic Photosensitive Member>

The electrophotographic photosensitive members obtained in Examples 1-1 to 1-20 and 2-1 to 2-20, and Comparative Examples 1-1 to 1-4 and 2-1 to 2-4 were evaluated as described below.

### [Evaluation Apparatus 1]

An evaluation was performed by mounting each of the electrophotographic photosensitive members produced in Examples 1-1 to 1-20 and Comparative Examples 1-1 to 1-4 on a copying machine imageRUNNER ADVANCE C5051 manufactured by CANON KABUSHIKI KAISHA.

Specifically, the above-mentioned evaluation apparatus was placed under a normal-temperature and normal-humidity environment having a temperature of 23° C. and a relative humidity of 50% RH, and each of the produced electrophotographic photosensitive members was mounted onto a process cartridge for a cyan color. The resultant was mounted onto the station of the process cartridge for a cyan color, and the evaluation was performed.

### [Evaluation Apparatus 2]

An evaluation was performed by mounting each of the electrophotographic photosensitive members produced in Examples 2-1 to 2-20 and Comparative Examples 2-1 to 2-4 on a reconstructed machine of a copying machine imageRUNNER ADVANCE DX C3830 manufactured by CANON KABUSHIKI KAISHA. Specifically, the above-mentioned evaluation apparatus was placed under a normal-temperature and normal-humidity environment having a temperature of 23° C. and a relative humidity of 50% RH, and each of the produced electrophotographic photosensitive members was mounted onto a process cartridge for a cyan color. The resultant was mounted onto the station of the process cartridge for a cyan color, and the evaluation was performed.

### (Evaluation of Abrasion Resistance)

The evaluation of abrasion resistance was performed by using the evaluation apparatus 1 and evaluation apparatus 2 described above. First, the initial total film thickness of the produced electrophotographic apparatus and electrophotographic photosensitive member was measured with a film thickness measuring device (kett). After the electrophotographic apparatus and the electrophotographic photosensitive member were left standing in an environment of a temperature of 23° C. and a humidity of 5% RH for 24 hours or more, the electrophotographic photosensitive member was mounted onto the cartridge for a cyan color of the electrophotographic apparatus. Next, an image having a print ratio of 5% was output in a cyan single color on A4-size plain paper, and 100,000 sheets were continuously output.

The thickness of the electrophotographic photosensitive member after the continuous printing was measured with the film thickness measuring device (kett), and the difference between the initial total film thickness and the film thickness of the electrophotographic photosensitive member after the continuous printing was calculated and adopted as an abrasion amount.

In the present disclosure, the calculated abrasion amount of 2 μm or less was judged to be a level at which the effect of abrasion resistance was obtained. In particular, an abrasion amount of 1.5 μm or less was judged to be an excellent level. Meanwhile, an abrasion amount of 2 μm or more was judged to be a level at which the effect of abrasion resistance was not obtained. The results of the evaluations of the electrophotographic photosensitive members produced in Examples 1-1 to 1-20 and 2-1 to 2-20, and Comparative Examples 1-1 to 1-4 and 2-1 to 2-4 each performed with the evaluation apparatus 1 and the evaluation apparatus 2 are shown in Table 2.

### (Evaluation of Image Smearing)

The evaluation of suppression of image smearing was performed by using the evaluation apparatus 1 and evaluation apparatus 2 described above. After the mounting of the produced electrophotographic photosensitive member on the copying machine, an image having a print ratio of 5% was output on 5,000 sheets of vertical A4-size paper in an environment of a temperature of 30° C. and a humidity of 80% RH, then power supply to the copying machine was stopped, the copying machine was paused for 48 hours, and a lattice image (4 lines, 40 spaces) and a character image in which hiragana characters “Iroha” were repeated (“Iroha” image) were output on vertical A4-size paper.

With regard to the resultant images, a suppressing effect on image smearing was evaluated according to the following evaluation ranks. In the present disclosure, ranks 5, 4, and 3 were judged to be levels at which the suppressing effect on image smearing was obtained. In particular, rank 5 was judged to be an excellent level. Meanwhile, ranks 1 and 2 were judged to be levels at which the suppressing effect on image smearing of the present disclosure was not obtained. The results of the evaluations of the electrophotographic photosensitive members produced in Examples 1-1 to 1-20 and 2-1 to 2-20, and Comparative Examples 1-1 to 1-4 and 2-1 to 2-4 each performed with the evaluation apparatus 1 and the evaluation apparatus 2 are shown in Table 2.
Rank 5: No image defects are observed in both the lattice image and the “Iroha” character image.
Rank 4: The lattice image is partially blurred, but no image defects are observed in the “Iroha” character image.
Rank 3: The lattice image is partially blurred, and the “Iroha” character image becomes partially faint.
Rank 2: The lattice image is partially lost, and the “Iroha” character image becomes entirely faint.
Rank 1: The lattice image is entirely lost, and the “Iroha” character image becomes entirely faint.

TABLE 2

| | Evaluation apparatus 1 | | | Evaluation apparatus 2 | |
|---|---|---|---|---|---|
| | Evaluation | | | Evaluation | |
| | of abrasion | Evaluation | | of abrasion | Evaluation |
| | resistance | of image | | resistance | of image |
| | (μm) | smearing | | (μm) | smearing |
| Example 1-1 | 1.95 | 5 | Example 2-1 | 0.85 | 5 |
| Example 1-2 | 1.60 | 5 | Example 2-2 | 0.95 | 5 |
| Example 1-3 | 1.28 | 5 | Example 2-3 | 0.45 | 5 |
| Example 1-4 | 1.13 | 5 | Example 2-4 | 0.55 | 5 |
| Example 1-5 | 1.00 | 5 | Example 2-5 | 0.50 | 5 |
| Example 1-6 | 1.05 | 5 | Example 2-6 | 0.58 | 5 |
| Example 1-7 | 0.98 | 5 | Example 2-7 | 0.40 | 5 |
| Example 1-8 | 1.28 | 5 | Example 2-8 | 0.38 | 5 |
| Example 1-9 | 1.35 | 5 | Example 2-9 | 0.55 | 5 |
| Example 1-10 | 0.70 | 5 | Example 2-10 | 0.33 | 5 |
| Example 1-11 | 0.73 | 5 | Example 2-11 | 0.40 | 5 |
| Example 1-12 | 0.63 | 5 | Example 2-12 | 0.30 | 5 |
| Example 1-13 | 1.53 | 5 | Example 2-13 | 0.63 | 5 |
| Example 1-14 | 1.30 | 4 | Example 2-14 | 0.60 | 4 |
| Example 1-15 | 1.15 | 5 | Example 2-15 | 0.58 | 5 |
| Example 1-16 | 1.75 | 5 | Example 2-16 | 0.78 | 5 |
| Example 1-17 | 1.43 | 5 | Example 2-17 | 0.60 | 5 |
| Example 1-18 | 0.73 | 4 | Example 2-18 | 0.28 | 4 |
| Example 1-19 | 0.68 | 5 | Example 2-19 | 0.30 | 5 |
| Example 1-20 | 1.33 | 5 | Example 2-20 | 0.65 | 5 |
| Comparative | 0.50 | 2 | Comparative | 0.28 | 2 |
| Example 1-1 | | | Example 2-1 | | |
| Comparative | 10.25 | 3 | Comparative | 7.75 | 3 |
| Example 1-2 | | | Example 2-2 | | |
| Comparative | 2.30 | 5 | Comparative | 1.03 | 5 |
| Example 1-3 | | | Example 2-3 | | |
| Comparative | 2.60 | 4 | Comparative | 1.30 | 4 |
| Example 1-4 | | | Example 2-4 | | |

According to one aspect of the present disclosure, the electrophotographic photosensitive member having high abrasion resistance and suppressing image smearing is provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

What is claimed is:

1. An electrophotographic photosensitive member comprising a surface layer containing a polymerized product of at least one kind of polymerizable charge-transporting compound selected from the group consisting of: a compound represented by the formula (CT-1); and a compound represented by the formula (CT-2),
wherein, in dynamic viscoelasticity measurement, the surface layer has a storage elastic modulus G′ and a loss elastic modulus G″ satisfying a relationship of G′>G″ at normal temperature, and has a peak top of Tan δ in a temperature range of 85° C. or more and 100° C. or less: 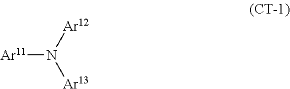
in the formula (CT-1), Ar¹¹ to Ar¹³ each independently represent a substituted aryl group or an unsubstituted aryl group, and a substituent that the substituted aryl group may have is an alkyl group having 1 or more and 4 or less carbon atoms, or a monovalent functional group represented by the formula (P-1), provided that the compound represented by the formula (CT-1) has at least one monovalent functional group represented by the formula (P-1): 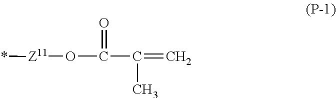
in the formula (P-1), Z¹¹ represents an alkylene group having 1 or more and 6 or less carbon atoms, and * represents a bonding site with a structure represented by the formula (CT-1) or (CT-2): 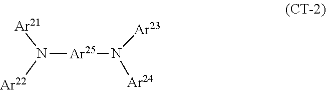
in the formula (CT-2), Ar²¹ to Ar²⁴ each independently represent a substituted aryl group or an unsubstituted aryl group, Ar²⁵ represents a substituted arylene group or an unsubstituted arylene group, a substituent that the substituted aryl group may have is an alkyl group having 1 or more and 4 or less carbon atoms, or a monovalent functional group represented by the formula (P-1), and a substituent that the substituted arylene group may have is an alkyl group having 1 or more and 4 or less carbon atoms, or a monovalent functional group represented by the formula (P-1), provided that the compound represented by the formula (CT-2) has at least one monovalent functional group represented by the formula (P-1).

2. The electrophotographic photosensitive member according to claim 1, wherein the storage elastic modulus G′ is 1,000 Pa or more, and the loss elastic modulus G″ is 1,000 Pa or more.

3. The electrophotographic photosensitive member according to claim 1, wherein the polymerizable charge-transporting compound is the compound represented by the formula (CT-1).

4. The electrophotographic photosensitive member according to claim 1, wherein the compound represented by the formula (CT-1) has 2 monovalent functional groups each represented by the formula (P-1).

5. The electrophotographic photosensitive member according to claim 1,
wherein the surface layer contains a polymerized product of a composition containing the polymerizable charge-transporting compound and a thiol compound, and
wherein, when a content of the polymerizable charge-transporting compound represented by each of the formulae (CT-1) and (CT-2) in the composition is represented by WCT, and a content of the thiol compound therein is represented by WT, the WCT and the WT satisfy the formula (1):
$$0.01 \leq WT / WCT \leq 0 \text{.07} . \qquad ( 1 )$$

6. The electrophotographic photosensitive member according to claim 5, wherein WT/WCT in the formula (1) is 0.01 or more and 0.03 or less.

7. The electrophotographic photosensitive member according to claim 5, wherein the thiol compound is a compound represented by the formula (T-1): 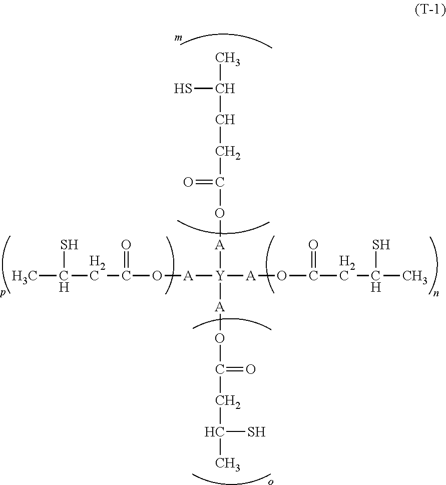
in the formula (T-1), Y represents a carbon atom, or a heterocycle having 2 or more nitrogen atoms, A represents a single bond, a hydrogen atom, or a linear alkylene group having 1 or more and 3 or less carbon atoms, “m”, “n”, “o”, and “p” each represent an integer of 0 or 1, and m+n+o+p is 2 or more.

8. The electrophotographic photosensitive member according to claim 7, wherein, in infrared absorption measurement of the surface layer, when an absorbance of a S—H stretching absorption band derived from the thiol compound represented by the formula (T-1) and showing an infrared absorption spectrum in a range of from 2,550 cm⁻¹ to 2,600 cm⁻¹ is represented by AT, and an absorbance of a C═O stretching absorption band of an ester group derived from the thiol compound represented by the formula (T-1) and showing an infrared absorption spectrum in a range of from 1,735 cm⁻¹ to 1,750 cm⁻¹ is represented by AE, the AT and the AE satisfy the formula (2):
$$AT / AE \leq 0.001 . \qquad ( 2 )$$

9. An electrophotographic photosensitive member comprising a surface layer containing a polymerized product of a composition containing a thiol compound, and at least one kind of polymerizable charge-transporting compound selected from the group consisting of: a compound represented by the formula (CT-1); and a compound represented by the formula (CT-2),
wherein, when a content of the polymerizable charge-transporting compound represented by each of the formulae (CT-1) and (CT-2) in the composition is represented by WCT, and a content of the thiol compound therein is represented by WT, the WCT and the WT satisfy the formula (1):
$$0.01 \leq WT / WCT \leq 0.07 \qquad ( 1 )$$ 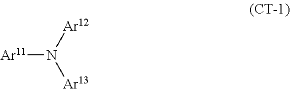
in the formula (CT-1), Ar¹¹ to Ar¹³ each independently represent a substituted aryl group or an unsubstituted aryl group, and a substituent that the substituted aryl group may have is an alkyl group having 1 or more and 4 or less carbon atoms, or a monovalent functional group represented by the formula (P-1), provided that the compound represented by the formula (CT-1) has at least one monovalent functional group represented by the formula (P-1): 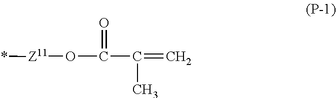
in the formula (P-1), Z¹¹ represents an alkylene group having 1 or more and 6 or less carbon atoms, and * represents a bonding site with a structure represented by the formula (CT-1) or (CT-2): 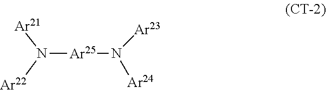
in the formula (CT-2), Ar²¹ to Ar²⁴ each independently represent a substituted aryl group or an unsubstituted aryl group, Ar²⁵ represents a substituted arylene group or an unsubstituted arylene group, a substituent that the substituted aryl group may have is an alkyl group having 1 or more and 4 or less carbon atoms, or a monovalent functional group represented by the formula (P-1), and a substituent that the substituted arylene group may have is an alkyl group having 1 or more and 4 or less carbon atoms, or a monovalent functional group represented by the formula (P-1), provided that the compound represented by the formula (CT-2) has at least one monovalent functional group represented by the formula (P-1).

10. The electrophotographic photosensitive member according to claim 9, wherein the thiol compound is a compound represented by the formula (T-1): 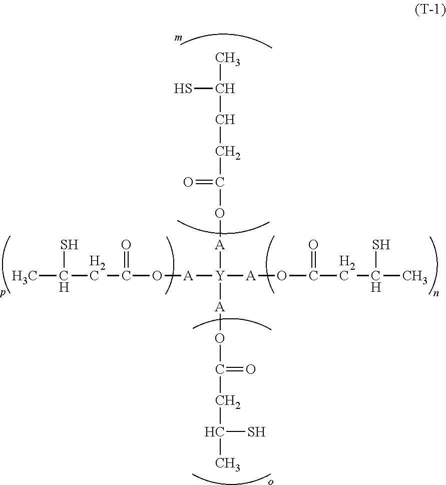
in the formula (T-1), Y represents a carbon atom, or a heterocycle having 2 or more nitrogen atoms, A represents a single bond, a hydrogen atom, or a linear alkylene group having 1 or more and 3 or less carbon atoms, “m”, “n”, “o”, and “p” each represent an integer of 0 or 1, and m+n+0+p is 2 or more.

11. The electrophotographic photosensitive member according to claim 1, wherein the surface layer contains a silicone compound having one of an acryloyloxy group or a methacryloyloxy group.

12. A process cartridge which is detachably attachable to a main body of an electrophotographic apparatus,
wherein the process cartridge comprises:
the electrophotographic photosensitive member of claim 1; and
at least one unit selected from the group consisting of:
a charging unit;
a developing unit; and
a cleaning unit, and
the process cartridge integrally supports the electrophotographic photosensitive member and the at least one unit.

13. An electrophotographic apparatus comprising:
an electrophotographic photosensitive member comprising a surface layer containing a polymerized product of at least one kind of polymerizable charge-transporting compound selected from the group consisting of: a compound represented by the formula (CT-1); and a compound represented by the formula (CT-2),
wherein, in dynamic viscoelasticity measurement, the surface layer has a storage elastic modulus G′ and a loss elastic modulus G″ satisfying a relationship of G′>G″ at normal temperature, and has a peak top of Tan δ in a temperature range of 85° C. or more and 100° C. or less: 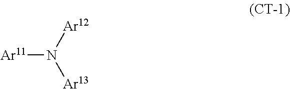
in the formula (CT-1), Ar¹¹ to Ar¹³ each independently represent a substituted aryl group or an unsubstituted aryl group, and a substituent that the substituted aryl group may have is an alkyl group having 1 or more and 4 or less carbon atoms, or a monovalent functional group represented by the formula (P-1), provided that the compound represented by the formula (CT-1) has at least one monovalent functional group represented by the formula (P-1): 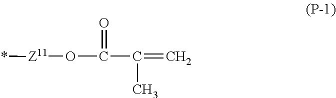
in the formula (P-1), Z¹¹ represents an alkylene group having 1 or more and 6 or less carbon atoms, and * represents a bonding site with a structure represented by the formula (CT-1) or (CT-2): 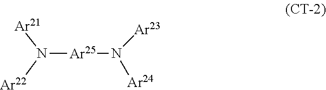
in the formula (CT-2), Ar²¹ to Ar²⁴ each independently represent a substituted aryl group or an unsubstituted aryl group, Ar²⁵ represents a substituted arylene group or an unsubstituted arylene group, a substituent that the substituted aryl group may have is an alkyl group having 1 or more and 4 or less carbon atoms, or a monovalent functional group represented by the formula (P-1), and a substituent that the substituted arylene group may have is an alkyl group having 1 or more and 4 or less carbon atoms, or a monovalent functional group represented by the formula (P-1), provided that the compound represented by the formula (CT-2) has at least one monovalent functional group represented by the formula (P-1); and
at least one unit selected from the group consisting of: a charging unit; an exposing unit; a developing unit; and a transfer unit.
